(19) United States
(12) Patent Application Publication
(10) Pub. No.: US 2024/0008736 A1
(43) Pub. Date: Jan. 11, 2024

(54) Multi-Modal System for Fluorescence and Reflectance Imaging

(71) Applicant: Manish Kulkarni, Pleasanton, CA (US)

(72) Inventors: Manish D Kulkarni, Pleasanton, CA (US); James Ogg, Sunnyvale, CA (US); Richard Graham, Livermore, CA (US); Noah Bareket, Saratoga, CA (US)

(73) Assignee: Netra Systems, Inc, Pleasanton, CA (US)

(21) Appl. No.: 18/028,514

(22) PCT Filed: Sep. 27, 2021
(86) PCT No.: PCT/US21/52260

§ 371 Date: Mar. 24, 2023

Related U.S. Application Data
(60) Provisional application No. 63/084,005, filed on Sep. 27, 2020.
(60) Provisional application No. 63/143,955, filed on Feb. 1, 2021.
(60) Provisional application No. 63/153,926, filed on Feb. 25, 2021.

(51) Int. Cl. A61B 3/10; A61B 3/12; A61B 3/15; A61B 5/00

(52) U.S. Cl. CPC A61B 3/102; A61B 3/1025; A61B 3/1241; A61B 3/152; A61B 5/7257; A61B 5/742; A61B 2503/40

(57) ABSTRACT

A multi-modal system that can perform optical coherence tomography (OCT), scanning laser ophthalmoscopy (fluorescence and reflectance), adaptive optics enhancement, and OCT-angiography simultaneously is presented. Such a system minimizes registration errors in multi-modality images, and accelerates biomedical research. Such a system could also permit enhanced high-speed diagnosis. This system would also permit a wide-selection of excitation wavelengths to measure fluorescence intensity from a variety of fluorophores and auto-fluorescing tissues. A novel animal positioning system that can easily align with the imaging beams for optimal image quality and high-speed experiment-setup and imaging of animals is also presented.

### CROSS REFERENCE TO RELATED APPLICATIONS

The instant application is a non-provisional application and claims priority to provisional U.S. patent application No. 63/084,005 titled “System for Fluorescence and Reflectance Imaging” filed on 27 Sep. 2020, provisional U.S. patent application 63/143,955 titled “ANIMAL STAGE AND MULTI-MODAL IMAGING SYSTEM” filed on Feb. 1, 2021 and provisional U.S. patent application No. 63/153,926 titled “ANIMAL STAGE IMAGING SYSTEM AND ALIGNMENT METHOD” filed on Feb. 25, 2021. This benefit is claimed under 35. U.S.C. § 119 and the entire disclosures of the Provisional U.S. patent Application Nos. 63/084,005, 63/143,955, and 63/153,926 are incorporated here by reference for all of their teachings.

### FIELD OF TECHNOLOGY

The following description relates to a system, and an apparatus for fluorescence imaging, detection and spectroscopy. The device also comprises OCT (Optical Coherence Tomography) imaging. The device can be used for diagnosis, imaging, measurements, evaluation or therapy. The device can be used for ophthalmic imaging and/or diagnosis, or retinal imaging and/or diagnosis. The apparatus and system can be used for the measurement and imaging of the eyes of the humans as well as the animals. The device can also be used for measurement/detection of other body parts of humans or animals. The device can also be used for measurement/detection of other living or non-living specimens.

### SUMMARY

The invention discloses a novel imaging/spectroscopy system, and apparatus for ophthalmology. The apparatus and system can be used for diagnosis, evaluation or therapy. The device can be used for ophthalmic imaging and/or diagnosis, anterior segment imaging and/or diagnosis, and/or retinal imaging and/or diagnosis. The apparatus and system can be used for the eyes of humans as well as animals. The device can also be used for the measurement/detection of other body parts of humans or animals. The device can also be used for the measurement/detection of other living or non-living specimens.

In an embodiment, a multi-modal system that can perform optical coherence tomography (OCT), scanning laser ophthalmoscopy (SLO) (fluorescence and reflectance), adaptive optics enhancement, and OCT-angiography simultaneously is presented. Such a system minimizes registration errors in multi-modality images, and accelerates biomedical research. Such a system could also permit enhanced high-speed clinical diagnosis for animals as well as humans.

In a few embodiments, this system would also permit a wide-selection of excitation wavelengths to measure fluorescence intensity from a variety of fluorophores and auto-fluorescing tissues.

In an embodiment, a novel animal positioning system that can easily align with the imaging beams for optimal image quality and high-speed experiment-setup and imaging of animals is also presented.

In an embodiment, the light source is split into at least 2 separate paths. One path would illuminate the OCT system and the second path would illuminate the SLO system. These two paths would have appropriate potentially variable filters to control the spectra of light entering into each imaging modality sub-system.

In one embodiment, the apparatus or system comprises of an ophthalmic system comprising of at least one means to hold the face of a patient (i.e., face-holder), a diagnostic component to perform diagnosis or evaluation of the eye or a therapeutic component for the treatment of the eye and a means to detect fluorescence.

In another embodiment, the apparatus or system comprises of a stage to hold an animal such as a mouse, rat, tree-shrew, squirrel monkey, dog, monkey, etc.

In another embodiment, the apparatus comprises of a face holder to hold the face of the animal. For example, the apparatus could hold the face of a dog, monkey, etc.

In another embodiment, the face-holder comprises of a resting pad to rest the chin (i.e., chin-rest).

In some other embodiments, the apparatus or the system comprises an optical coherence tomography (OCT) imaging.

In some other embodiments, the apparatus or system comprises an optical coherence tomography (OCT) imaging apparatus/system and the OCT apparatus/system comprises a spectrometer to implement spectral-domain OCT.

In some other embodiments, the system apparatus comprises of optical coherence tomography (OCT) imaging apparatus/system and the OCT apparatus/system comprises of a tunable wavelength (or frequency) light source to implement swept-source OCT or optical frequency domain reflectometry (OFDR).

In some other embodiments, the apparatus or system comprises of optical coherence tomography (OCT) imaging apparatus/system and the OCT apparatus/system comprises of a depth-scanning reference mirror to implement time-domain OCT.

In one embodiment, the OCT system and apparatus mentioned above comprises of a light source of specific bandwidth, isolator, beam splitter, optical delivery unit, specimen, a grating, a detector array and a processor containing specific algorithms for signal and/or image processing.

In another OCT embodiment, as an additional feature, a polarization compensator is added to the basic configuration mentioned above. In one embodiment, a fiber stretcher is added in the basic configuration. The fiber stretcher is used to adjust the path-length in the corresponding arm of the system.

In one embodiment, the system comprises of an OCT system comprising of a light source that provides a broadband light (of specific bandwidth) for acquiring an image from the subsurface area of a specimen. The specimen may be, but not limited to a moving sample, a stationary sample or a combination of both. The specimen may be a human or an animal eye or a device similar to an eye or a test device emulating an eye. In another embodiment, the system is modular so that a user can add off-the-shelf products to enhance the system capabilities. In another embodiment, several combinations of the basic configuration and additional components may be added to enhance the performance of the apparatus as a system as shown in the various figures that accompany this application, but not limited to only those.

In another embodiment, specific algorithm(s) reside in a processor in the system to create an OCT image including OCT-derived enFace image or cross section. The processor uses the algorithms such as the frequency resampling, demodulation, dispersion compensation, OCT angiography and Doppler processing to produce highly sensitive and high quality images. In another embodiment, the system performs spectroscopic detection. The resultant spectra are analyzed by the processor using inverse Fourier transformation and relevant signal processing for obtaining depth dependent (i.e. axial) reflectivity profile called A-scan. In another embodiment, two dimensional tomographic images, B-scans, are created from a sequence of axial reflectance profiles acquired while a beam is moved across an area of interest in the specimen.

In one embodiment, the system may comprise of an OCT/OCDR (optical coherence domain reflectometry) sub-system comprising of a light source, an isolator, a processor, a fiber stretcher, a source arm, a reference arm, a sample arm, a detection arm, a beam splitter, a detector array, a grating unit, an optical delivery unit which can be, and a specimen (e.g., an eye) for analysis.

In some embodiments, the bulk of the OCT/OCDR sub-system, e.g., light source, an isolator, a processor, a fiber stretcher, a source arm, a reference arm, a sample arm, a detection arm, a beam splitter, a detector array or optical detector(s), a grating unit resides in the base of the system.

In another embodiment, an OCT/OFDR (optical frequency domain reflectometry) sub-system may comprise of a tunable light source, an isolator, a processor, a fiber stretcher, a source arm, a reference arm, a sample arm, a detection arm, a beam splitter, a detector, an analog-to-digital converter, an optical delivery unit, and a specimen (e.g., human or animal eye) for analysis. In some embodiments, a polarization compensator may be used on the sample and/or reference arms of the OCT interferometer.

In another embodiment, the OCT/OCDR sub-system enables a user to adjust the reference arm and the sample arm in order to adjust the path-lengths to detect the specimen depth location and OCT back-scattered signal and/or polarization of the light beam to get a better quality image (improved signal to noise ratio).

In another embodiment, light from a broadband light source operating at a suitable center wavelength is sent to an isolator, and then to the beam splitter using the source arm of the OCT sub-system. In another embodiment, the beam splitter splits the broadband light into two parts. A fraction of the light beam goes to the reference mirror using the fiber stretcher (on the reference arm) and other beam goes to the specimen using the sample arm.

In some embodiments, the apparatus/system comprises of the means for an eye-fixation target.

In some embodiments, the apparatus/system comprises of fiber or cables running from the instrument to the eye.

In some embodiments, the apparatus/system comprises of a screen to display measurement or imaging results.

In some other embodiments the display screen is a touch-sensitive screen.

In some embodiments, the apparatus/system evaluates or scans the retina and/or the posterior segment. In some other embodiments, the apparatus/system evaluates or scans the cornea and/or anterior segment.

Ophthalmic imaging of mice and small animals in a research environment typically requires a series of animals to be rapidly positioned for imaging with minimal adjustments required between animals, and minimal adjustments required to image the retina from different angles. Ensuring that the retina does not move out of view as the animal is rotated through alternate viewing angles reduces the time required for imaging an individual animal. Changing either the eye or individual animal under observation such that minimal adjustment is needed to re-center the eye at the working point of the imaging system reduces the time required for acquiring data from a series of subjects.

In an embodiment, a mouse positioner and cartridge support the primary functionality of the system by positioning and stabilizing the mouse or the subject or the animal. A conceptually equivalent positioner could be applied to other small animals such as rats, shrews, etc. Many aspects of the positioner described here could also be applied to other small animals, such as fish, etc

Quarter-wave plate (QWP) crown: In an embodiment, this cylindrical fitting allows the rotation of a QWP while providing mounting threads for lens caps and test targets. The quasi-cylindrical pocket for the QWP has a flat surface which aligns with the fast axis of the QWP. The outer surface has a matching machined flat, which serves as an indication of the angle of the QWP's fast axis. A plastic-tipped setscrew can be tightened to secure the crown and QWP at a set angle.

Deformable mirror (DM) adapters: In another embodiment, DM adapters are used. The DM mounting uses adapters specific to DMs from multiple sources. The adapters compensate for differences in mirror plane position and mounting features of the various DMs. This permits multiple DMs and a typical mirror, and paired adapters, to be interchanged without major modification or realignment to the system.

Dark vents: These permit airflow while limiting light intrusion. Mounting features are matched to a modular cable pass-through, so that either cable routing or airflow routing could be configured without needing to replace the dark box walls. See FIG. 6.

Coarse alignment cameras: Coarse alignment of the mouse to the objective can be aided by dual cameras, each aligned to the working point of the objective. These cameras would be orthogonal or quasi-orthogonal with respect to each other and the objective. When the mouse eye was centered in both cameras, it would also be centered to the objective at the working distance. See FIG. 10.

These cameras might be mounted to the objective, to the chassis, to the subframe, or to the table. They might be secured by a mechanical armature, suction, and/or magnets.

These cameras can be mounted to give a better viewpoint than the user would comfortably have.

These cameras might be sensitive to NIR, permitting course alignment of the mouse without visible excitation. Specific mouse protocols limit the exposure of the mouse's eyes to light for experimental purposes. More generally, eyes are more susceptible to damage from visible wavelengths.

Alternatively, such a camera can be used singly.

Such cameras could be used with a scanning pattern that will aid coarse alignment, such as cross-hairs or a circle.

Linear variable filters module: To permit a continuous color range of fluorescence and reflectance excitation and detection, a number of linear variable filters and dichroics can be clustered together, analogous to the discrete filters and dichroics of a fluorescence filter cube.

Furthermore, this linear variable filters module can be engineered to have common mounting features with a stage of fluorescence filter cubes. These common mounting features would result in the linear variable filters module being interchangeable with the fluorescent filter cube module. See FIG. 4.

These linear variable filters can be cascaded to give a sharper transmission cutoff than individual filters offer. Like the clustered filters, the cascaded filters can be fabricated with matched curves, and aligned to each other with a static offset. This would permit them to be driven by a single motor. Alternatively, they might be mounted to separate motors for individual control.

The positioning of such cascaded filters would need to be carefully adjusted. The wavelength cutoffs of the linear variable filters vary along the length of these filters. The sharper cutoff from cascaded filters requires both filters to be positioned so that they cut off equally. If the cutoff is driven by one filter or the other, the combined cutoff won't be sharper than the cutoffs of the individual filters.

This module could be fitted with lenses to focus the light near the filters and./or dichroic, reducing the spot size on the filters and dichroic. Importantly, this reduced spot size would reduce the effect of variations in the 50% transmission wavelength and deviations from flatness across the spot.

There would need to be some provision for the blockage of stray light, such as black barriers. Conventional filters transmit or block light uniformly across their surface. In contrast, the transmission of light varies along the length of linear variable filters. Light that should be blocked could leak through due to being offset towards one end of the filter or the other.

If mounted to separate motors, the filters and dichroics can be inverted relative to each other. For example, the dichroic could be mounted so that the 50% transmission wavelength decreased top to bottom, while the emission filter could be mounted so that the 50% transmission wavelength increases top to bottom. This limits leakage from getting past both filters by being either high or low.

Alternatively, the filters can be non-inverted, but mounted on alternate sides of a focal point.

Alternatively, this module might use exponentially variable filters.

Multiple independent stages: A wide range of operating modes can be implemented with multiple, independent stages. In one example, a first beam splitter stage might be able to switch between clear plates, mirrors, and dichroics to implement OCT-only operation, SLO-only operation, or combined OCT and SLO operation, while a fluorescent filter cube stage might be able to switch between multiple sets of filters and dichroics, with the two stages together offering a large number of options while each stage separately only offers a limited number of positions. In a second example, the fluorescent filter cube stage might be replaced with a set of linear variable filters and dichroics.

Shutters: The enclosure can be fitted with light-blocking shutters. These shutters might be mechanically controlled, for example, to prevent exposure of sensitive optical elements to light when the system is powered down and the cover removed for service. Alternatively, these shutters might have rapid electrical control, for example, which would permit them to prevent a localized area of the eye to be exposed to high-power light and to prevent ocular exposure over the MPE (maximum permissible exposure) in the event of failure of the scanning mirror(s).

Variable focal length lenses (VFLs): A single VFL can be used to electronically make small adjustments in the focal point of the objective. This can be utilized to automate the three-way optimization of focal point, reference arm length, and software dispersion compensation.

A second VFL can be used to compensate for chromatic aberrations. As some implementations of the system will operate on variable wavelengths, the chromatic aberrations can be expected to vary as a result. This second VFL can be used to manually or automatically adjust the compensation for chromatic aberration.

Broadband excitation and adjustable excitation filters: This system can utilize a single broadband source that covers the entire spectrum necessary for excitation, along with filters that can be set to block the undesired bands for a specific configuration. Alternatively, it can use two broadband excitation sources, for example, one to provide visible light and one to provide NIR, along with multiple adjustable filters. Alternatively, it can combine broadband sources and discrete narrow band sources, along with suitable adjustable filters. For example, the system could use a collection of super-luminescent diodes (SLDs) and a selection of multiple lasers to provide the necessary combinations of broadband and narrow-band light as needed for specific scans.

The VFL can change its focal length by using electrical signals, pressure signals, temperature signals, acousto-optic modulation, etc.

The system can be used to image mice, rats, other small animals, zebra-fish, tadpoles, mid-sized and large animals, primates, dogs, and humans. This system could also be used to image non-living specimens, test objects, test materials, excised tissues from animals and humans.

This system can be used to optionally combine OCT (optical coherence tomography), fluorescence imaging (and/or SLO, scanning laser ophthalmoscope), OCT-angiography, Doppler OCT, adaptive optics OCT and/or adaptive optics SLO and/or adaptive optics Doppler OCT and/or adaptive optics), and/or adaptive optics OCT-angiography.

These modes can utilize non-overlapping wavelength bands. For example, SLO and OCT can be combined by allocating a shortest wavelength range for fluorescence excitation and SLO reflectance signals, an intermediate wavelength range for fluorescence SLO detection, and a higher wavelength range for OCT. These bands might typically involve the visible excitation and emission bands of a fluorophore of interest (e.g. GFP), and NIR for OCT. Alternatively, these bands can be divided. For example, the system might use a broadband visible light source and a fluorescence filter cube engineered for use with multiple, non-overlapping excitation and emission bands to detect fluorescence from multiple fluoroscopes in a single scan. The system could be equipped with multiple fluorescence channels. Alternatively, with a single fluorescence channel, each fluorophore can be individually scanned by adjusting the excitation filter at the light source to selectively excite individual fluorophores. Alternatively, the wavelength bands can be allocated in other ways. For example, a main NIR band can be utilized for OCT as well as excitation for infrared fluorophores, with a longer-wavelength band allocated for fluorescence SLO detection. (Called split-spectrum mode.)

In one embodiment, the multiple imaging results, e.g. OCT, OCT angiography, SLO reflectance, SLO fluorescence, etc, can be displayed in separate views, e.g. displaying an averaged OCT en-face image alongside a differential view indicating flow via OCT angiography.

In another embodiment, multiple imaging results can be superimposed by color, such as overlaying a grayscale averaged OCT en-face view with an indication of flow inferred from OCT angiography scaled between transparent and red. Similarly, a clear-to-green image indicating fluorescence can be superimposed upon a grayscale OCT en-face image.

In another embodiment, multiple imaging results can be presented serially, e.g. fluorescence results can be sequentially presented with reflectance results, with the display fading cyclically between the reflectance results colored to approximate the an excitation wavelength of the fluorophore and the fluorescence results colored to approximate an emission wavelength of the fluorophore.

Adaptive optics can be sensorless or based on wavefront-sensors (WFS) Adaptive optics could be completely digital (without using any deformable mirrors or wavefront controllers).

### BRIEF DESCRIPTION OF THE DRAWINGS

FIG. 1 is a block diagram of an OCDR-OCT sub-system 100 (which can be incorporated into an ophthalmic system), in accordance with the prior art; the key elements being a grating unit, a fiber optic mirror, and a fiber stretcher.

FIG. 2 is a block diagram of the multi-modal-OCDR-OCT-SLO system 200 (which can be incorporated into an ophthalmic system), in accordance with an embodiment of the present invention; the key elements being 2 VFL (variable focal-length-lenses), pinholes, fluorescent filter cubes (FFC) and a fiber stretcher.

FIG. 3 shows a motor-driven fluorescent filter cube stage with multiple, configured to use fluorescent filter cubes

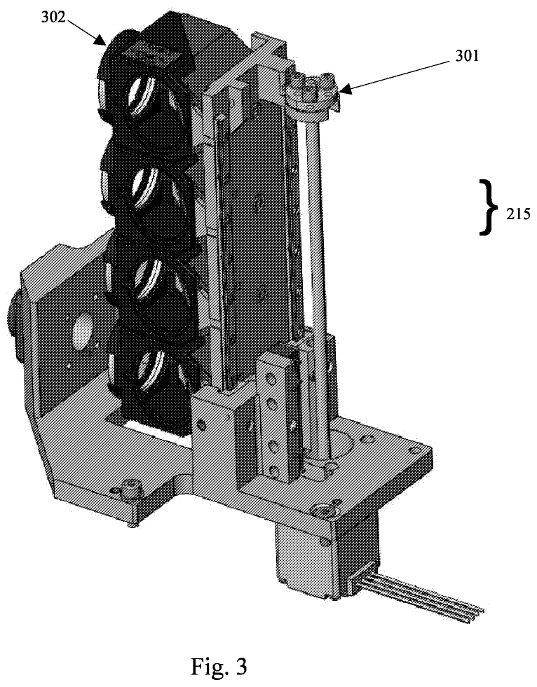

FIG. 4 shows a motorized module of linear variable excitation filters, emission filters, and variable dichroic, as well as lenses to focus the beam near the filters and dichroic.

FIG. 5 shows an integrated first beam splitter module that combines a second beam splitter, an optional wavefront sensor, an optional variable focal length lens, and optional galvanometric mirrors.

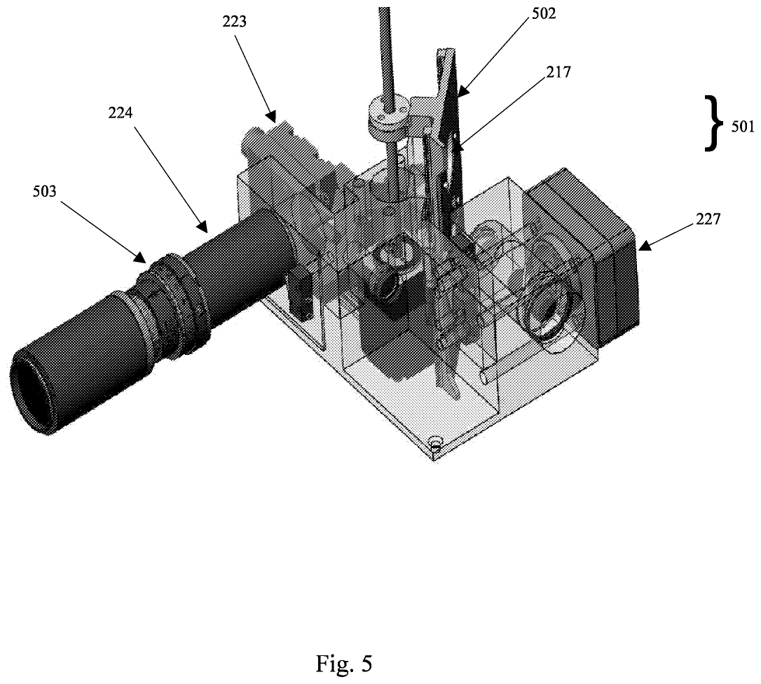

FIG. 6 shows dark vents and modular cable pass-throughs.

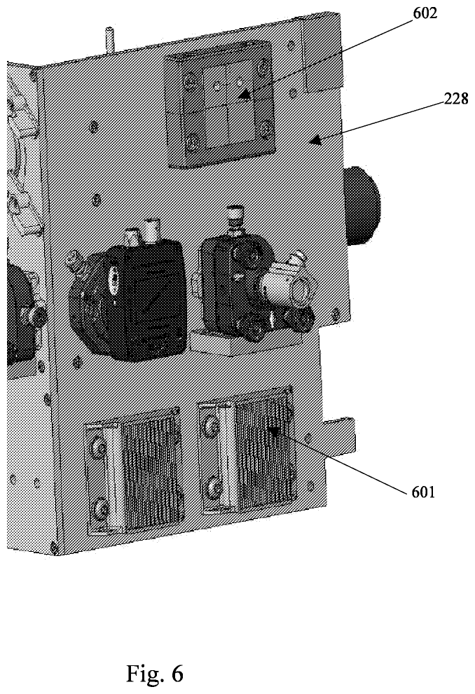

FIG. 7 shows a mouse positioner with 8 precise degrees of freedom plus engage/disengage via a sliding base and travel stop. The stop is not shown.

FIG. 8 shows an angled mouse cartridge. It is a 3D-printed cartridge with ambidextrous mounting features, integrated palate bar, and jaw depressors

FIG. 9 shows a muzzle retainer secured with spring clips to the cartridge

FIG. 10 shows coarse alignment cameras.

FIG. 11 shows an alignment ball tool for the 8 degree of freedom (left) mouse positioner and a lens practice target (right).

FIG. 12 shows spheres aligned to be objective to scan a surface (right) or trivially to scan a spot (left) and the resulting B-scans

FIG. 13 shows the effect of eye or lens misalignment on B-scan

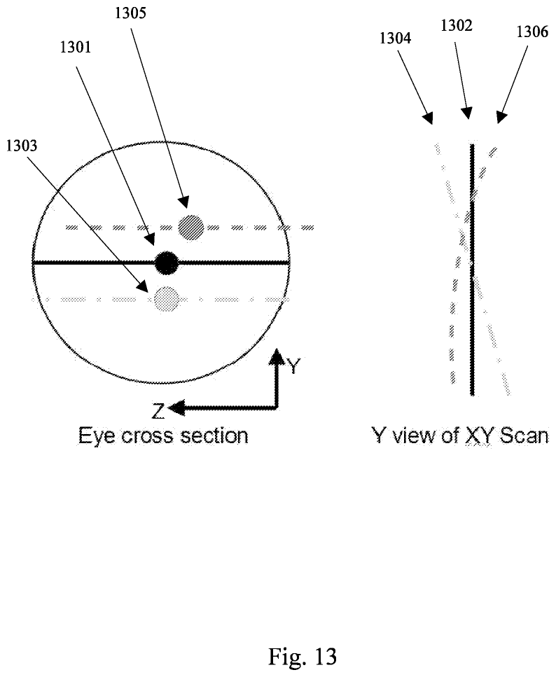

FIG. 14 shows an alternate cartridge embodiment that is single-sided, with a bite bar and routing for an elastic muzzle strap. Arrows indicate the routing of the elastic muzzle strap.

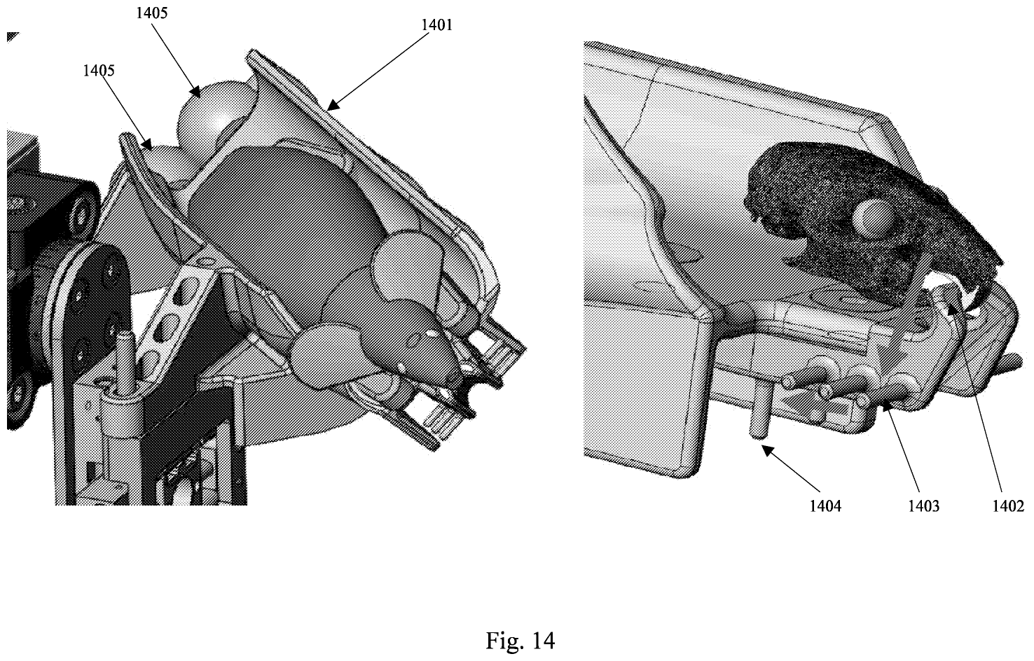

FIG. 15 shows an alternate cartridge embodiment with support for gas anesthesia provided by a machined manifold with an outlet near the nose and dual inlets under the jaw. A wide muzzle strap, to contain anesthesia gasses, is secured by thumbscrews. Arrows indicate the airflow paths.

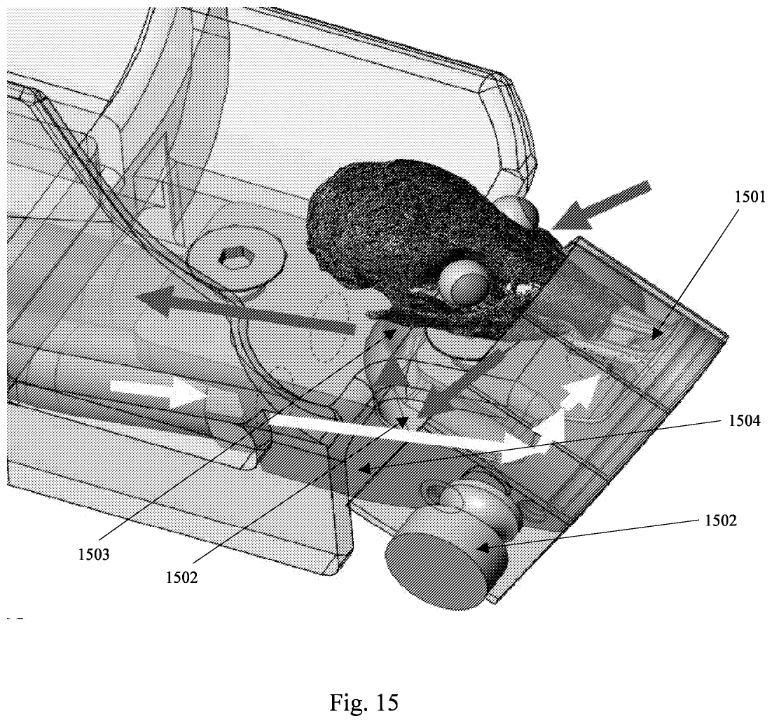

FIG. 16 shows an alternate cartridge mounting arm configuration to reduce snagging during placement and removal.

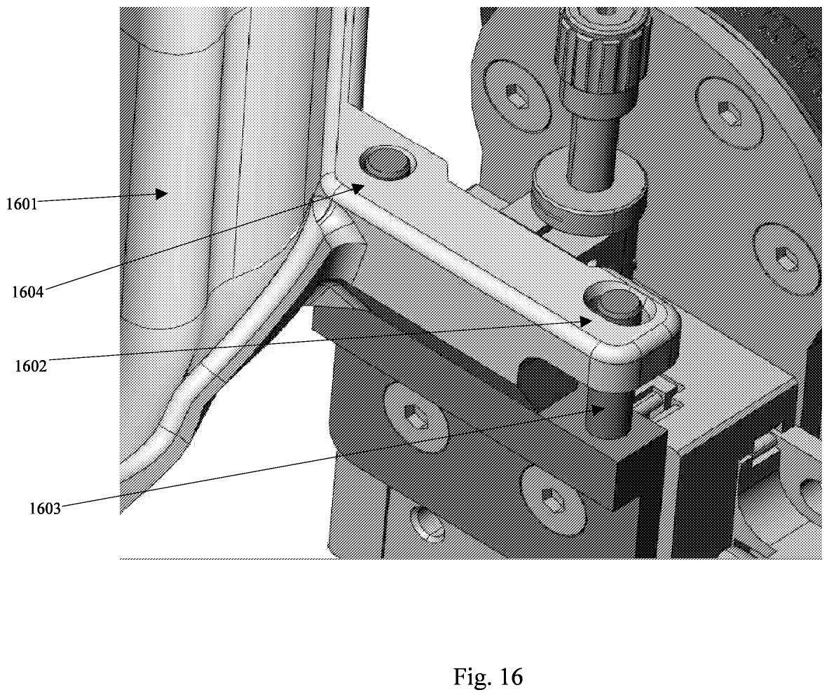

FIG. 17 shows an example of a 4-layer test target with metalization pattern for characterizing imaging performance and holes for supporting fluorescent particles embedded in a polymer and axially co-located with each metalization layer.

FIG. 18 shows an example schematic of a confocal reflectance detector.

FIG. 19 shows an example of a ball-lens target with one side supporting a mult-layer structure emulating multiple layers and an absorptive optic-nerve analog.

FIG. 20 is a view of a first step in the procedure for using an alignment ball tool 1101 showing nasal/temporal and superior/inferior rotations both rotated to 0°.

FIG. 21 is a view of a step in a procedure for using an alignment ball tool 1101 showing nasal/temporal and superior/inferior rotations both rotated to 180°.

FIG. 22 is a view of a first step in a procedure for using an alignment ball tool 1101 showing nasal/temporal rotation rotated to 0° and superior/inferior rotation rotated to 90° degrees.

FIG. 23—is a simple method to perform sensorless adaptive optics

FIG. 24—flowchart describing the OCT image formation using OFDR or OCDR technologies

FIG. 25—flowchart describing Doppler OCT imaging

FIG. 26—is a flow chart of method of demodulating the signal to recover the complex envelope of the OCT/OCDR/OFDR signal.

FIG. 27—is a flowchart of method of Doppler processing the signal to estimate the Doppler shift and the corresponding velocities of the particles in the specimen.

### DETAILED DESCRIPTION

The instant disclosure describes a technological advancement of an ophthalmic apparatus and system.

In some embodiments, the system comprises a means to detect fluorescence from fluorophores or autofluorescence within an eye or any other specimen.

In some embodiments, the light source for OCT can act as an excitation light source for fluorescence.

In some embodiments, a common light source with very broad bandwidth can be split using a system of optical filters into independent beams for OCT imaging and SLO fluorescence/reflectance imaging.

In some embodiments, there is a specific source that can act as an excitation light source for fluorescence.

In some embodiments the wavelength of the excitation light can be tuned over a wide range, e.g., 300-900 nm.

In some embodiments, the intensity of the excitation light can be tuned/varied. In some embodiments the intensity can be tuned/varied over a wide range as well.

In some embodiments, the fluorescence measurement is facilitated by passing the light through a fluorescence filter, which is a high-pass or band-pass filter.

In some embodiments, the pass-band of the fluorescence filter is selected such that the wavelength (or spectrum) of the excitation source is outside the pass-band of the fluorescence filter.

In some embodiments, the pass-band of the fluorescence filter is tunable. This may include tuning the high-pass-cut-off wavelength. If the fluorescence filter is a band-pass filter, then the bandwidth and/or upper cut-off wavelength and/or lower cut-off wavelength can be tuned.

In some embodiments, the pass-band of the fluorescence filter and the excitation wavelength (or spectrum) are tuned such that the wavelength (or spectrum) of the excitation source is outside the pass-band of the fluorescence filter.

The excitation light can be a tunable laser or light emitting diode or a tunable broad-band source. The excitation light could be a broad-band light followed by a tunable filter (termed excitation filter). The pass-band of the excitation filter is tunable. This may include tuning the short-pass-cut-off wavelength. If the excitation filter is a band-pass filter, then the bandwidth and/or upper cut-off wavelength and/or lower cut-off wavelength can be tuned.

In some embodiments, the pass-band of the fluorescence filter and pass-band of the excitation filter are tuned such that the passband of the excitation filter does not overlap with the pass-band of the fluorescence filter.

In some embodiments, the imaging system comprises of OCT simultaneous with fluorescence imaging.

In some embodiments, OCT light source excites the fluorescence in the specimen.

In some embodiments, the OCT system comprises of tunable light source.

In some embodiments, the fluorescence is created and measured using a scanning laser imaging system. If the specimen is an eye, the system is called scanning laser ophthalmoscope.

In some embodiments, the fluorescence is detected using a detector.

In some embodiments, the fluorescence is imaged using a 2-dimensional sensor. This sensor can be a 2-D array.

In some embodiments, the excitation light illuminates an area on the specimen. The specimen can be an eye or some other specimen.

In an embodiment, the excitation light is filtered using a monochromator. Other examples of tunable filters include liquid crystal tunable bandpass filter, acousto-optic-tunable filters, scanning Fabry-Perot filters or interferometers, and scanning monochromators. One could also use continuously variable filters (or linear variable filters). One could tune the pass-band (or cut-off-wavelengths) of the filters by tilting the filters at appropriate angles. These tilting tunable filters include tunable long-pass-filters, tunable short-pass filters and tunable band-pass filters.

In an embodiment, the fluorescence filter can reside in front of the detector or sensor or camera.

In another embodiment, the excitation filter can reside in front of the light source.

The diagnostic, evaluation and/or therapeutic components can direct light to the eye (and receive the back-scatter from the eye) using an optical delivery unit (which comprises of various opto-mechanical elements to direct light to a target and collect retro-reflected or backscattered light). In some embodiments, the optical delivery unit is a part of the face-holder.

In an embodiment, the fluorescence filter and/or the excitation filter can reside in the optical delivery unit.

In some embodiments, the retro-reflector might be a corner cube, ‘cat's-eye’ retroreflector or similar configuration.

In some other embodiments, the apparatus/system comprises of an eye-piece which is optics and mechanics used to evaluate or treat the eye. In some embodiments, the eye-piece is a part of the optical delivery unit.

In an embodiment, the fluorescence signal is directed towards a spectrometer to measure fluorescence spectra.

In another embodiment, the fluorescence signal is processed to compute fluorescence lifetime. This could lead to fluorescence lifetime imaging or fluorescence lifetime imaging microscopy or time-domain fluorescence imaging.

In one embodiment, the fluorescence life-time is measured by sending a pulsed excitation light source to the specimen.

In some embodiments, the apparatus/system comprises of a screen to display measurement or imaging results. Thus, the display can host diagnostic-assisting results. In some other embodiments the display screen is a touch-sensitive screen. In some other embodiments, the display could have 3-D capabilities (or stereoscopic capabilities) showing 3-dimensional features of the data or the measurements or anatomic features.

In some embodiments, the apparatus/system comprises of a keyboard to control the apparatus/system. The keyboard can optionally comprise of a mouse or a controlling ball or a joystick. In some other embodiments the keyboard is a touch-sensitive screen. In some embodiments, the touch-sensitive display comprises of the keyboard.

In some embodiments, the eye-piece is shifted using a precision slide to evaluate or treat the left or right eye. In some other embodiments, the eye-piece is shifted using a sliding rod. In some embodiments, the eye-piece is positioned using a micro-precision slide. In some embodiments, the eye-piece is a part of the optical delivery unit.

In some embodiments, the eye-piece has railings to move it forward and/or backward with respect to the patient's or subject's eye.

In some embodiments, the apparatus/system comprises of the means for an eye-fixation target. These means could comprise of a display inside the eye-piece. The display could have an eye-fixation target as desired by the operator of the apparatus/system.

In some embodiments, the apparatus/system could comprise of a projector (sometimes termed pico-projector) to display the results or the images or the data on a wall or a screen.

In some embodiments, the apparatus is used for ophthalmic imaging. Ophthalmic imaging includes (but does not limit to) retinal imaging and anterior segment.

### OCT/OCDR/OFDR Sub-System Description

In some other embodiments, the ophthalmic apparatus/system comprises of optical coherence tomography (OCT) imaging. Optical coherence domain reflectometry (OCDR) is a 1-dimensional measurement system and OCT is a 2-D extension of OCDR. Since OCT and OCDR are similar, sometimes we would refer these as OCT-OCDR systems. The diagnostic components or systems based on OCT-OCDR, will be called as OCT-OCDR based diagnostic components.

Optical coherence tomography (OCT) and OCDR are very similar to ultrasound imaging. OCDR-OCT provides cross-sectional images of micro-features that are acquired from adjacent depth resolved reflectivity profiles of the tissue. OCT also employs a fiber optically integrated (or a free-space) Michelson interferometer illuminated with a short coherence length light source such as a superluminiscent diode (SLD). The interferometric data are processed in a processor/computer and displayed as a gray-scale or false-color image. In an OCDR-OCT image, the detectable intensities of the light reflected from human tissues range from 10⁻⁵ to 10⁻¹¹th part of the incident power.

In some other embodiments, the apparatus or system comprises of optical coherence tomography (OCT) imaging apparatus/system and the OCT apparatus/system comprises of a spectrometer to implement spectral-domain OCT.

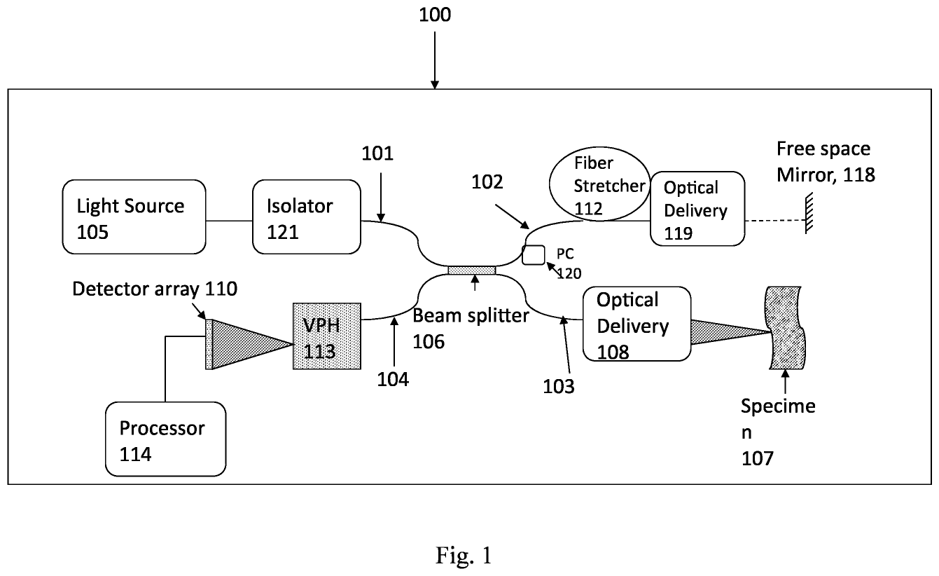

OCDR-OCT System: FIG. 1 shows an OCDR-OCT system 100 comprising of a light source 105 of a specific bandwidth, isolator 121, processor 114, fiber stretcher 112, source arm 101, reference arm 102, sample arm 103, detection arm 104, beam splitter 106, detector array 110, a grating unit 113, optical delivery unit 108, fiber optic mirror 117 and a specimen 107 (could be a human or an animal eye) for analysis.

In some embodiments, the optical delivery unit 108 is further integrated with the face-holder of the ophthalmic system.

A light source 105, in a system or as a part of the apparatus/system.

The center wavelength (λ₀) for the retinal applications range from 750 nm till 1050 nm. Visible wavelengths (e.g., 300-800 nm) could also be used. Water (and aqueous humor) absorption is minimal for this wavelength range. The power for retinal applications ranges from 0.1 mW to 10 mW. Per ANSI safety standards only are permitted incident on the eye at this wavelength range of 750 nm till 850 nm. The center wavelength most ideal for the non-retinal applications (e.g., skin, anterior segment of the eye, gastrointestinal tract, lungs, teeth, blood vessels, subsurface area of semi-conductors, chip manufacturing, sensitive medical equipment's etc.) range from 1050 nm till 1350 nm. Visible wavelengths (e.g., 300-800 nm) could also be used. The longer wavelength is more suitable for thick scattering tissues since scattering is less at higher wavelengths. The system depth resolution (DR) is inversely proportional to the FWHM spectral width (or bandwidth Δλ) of the light source spectrum. It is given by the following equation:

$$DR = \frac{2 ln 2}{π} \frac{{λ}_{0}^{2}}{Δ λ} \qquad ( Eq 1 )$$

The full-width-half-max (FWHM) spectral width of the light source typically ranges from 10 nm till 150 nm. The power for non-retinal applications ranges from 0.1 mW till 30 mW in the wavelength range from 1050 nm till 1350 nm. The full-width-half-max (FWHM) spectral width of the light source typically ranges from 10 nm till 150 nm.

The light source 105 may be electrically operated. These can be battery operated while in transit. The forward voltage typically ranges from 2 to 10 Volts. The forward current typically ranges from 100 mA to 1 A. Some of these sources need to be thermo-electrically controlled (TEC). The operating internal temperature for some sources is typically 25° C. The corresponding thermistor resistance is 10 kilo-Ohms (10 kΩ). Typical TEC current is 1.5 A. Typical TEC voltage is 3-4V. The light source may also be a tunable light source as shown in other system/apparatus embodiments.

The isolator 121 protects the light source from back reflections and permits the transmission of light in the forward direction with a limited loss. The fiber-optic isolator used in device would need to operate on a broad range of spectrum to cover the full spectral-width of the light source (depending upon the source spectral shape, typically 2*FWHM bandwidth Δλ). Thus the operating wavelength range is λ₀+/−Δλ. Typical isolation is 20-40 dB, and insertion loss is 0.5-3 dB. The polarization dependent loss is typically 0.5 dB or less. Return loss is typically more than 40 dB.

The isolator 121 comprises of an input linear polarizer, a (λ/8) Faraday rotator or a waveplate, and an output linear polarizer. The (λ/8) Faraday rotator or a waveplate rotates the light transmitted through the input polarizer by 45 degrees. The output polarizer needs to have the same direction as “the input polarizing direction rotated by 45 degrees” in order to have the maximum transmission and maximum isolation. The light returning to the isolator from the remaining system gets linearly polarized by the output polarizer and is rotated by 45 degrees, making it orthogonally polarized as compared to the input polarizing direction. Thus, the returning light is totally absorbed.

Fiber stretcher 112 comprises of a fiber looped around a piezoelectric device (which is a solid block that can be expanded or contracted by electric voltage). The fiber stretcher is not strictly necessary in an OCDR/OCT system, it can be optionally used. The purpose of a fiber stretcher is to increase or decrease the path-length in the interferometer by increasing or decreasing the fiber-length. Although the fiber stretcher 112 is shown in the reference arm, it can be placed ether in the reference arm or sample arm. If the fiber stretcher 112 is kept in the reference arm, since the fiber is looped around the piezoelectric device, care must be taken to provide extra fiber in the sample arm so that the sample arm and reference arm path lengths are matched.

The fiber optic mirror can be situated on the tip of the fiber (not shown).

The optical delivery unit 108 can be attached to the face holder in some embodiments.

Detector array 110 is a line-scan camera. It has typically 1024-4096 pixels, though the proposed embodiment is not limited to these numbers. Typically it is a CCD or CMOS camera. Line-rate (rate of acquisition of arrays) is typically 10000 lines/s to 400000 lines/s, though the proposed embodiment is not limited to these numbers. Each pixel outputs a value which typically has an 8-bit or 12-bit format, though the proposed embodiment is not limited to these numbers. The pixel size is typically 14 microns (height) and 14 microns (width). The light dispersed by the grating is focused on the detector array to generate the light spectrum. The output of the array (line-scan camera) is typically directed to the computer using an Ethernet cable (e.g., Gigabit Ethernet) or a USB (typically 2.0 or 3.0) cable, etc. The operating wavelength ranges from 400 nm to 1100 nm for retinal applications. The above numbers and examples are given for illustrative purposes only, the proposed embodiment is not limited to these numbers or examples.

The beam splitter 106 (made of fiber optics) splits the light typically into 50/50. It is built using two fused single-mode fibers. The fiber for retinal applications (˜800 nm wavelength) has 4-6 microns core diameter and 125 microns cladding diameter, 0.130 core numerical aperture (NA), cutoff wavelength of typically 730 nm. The insertion loss (in addition to designed 3 dB or 50% loss) is typically 0.3 dB. For the couplers used for OCT, the length of the fiber in the reference and sample arms is very important and the lengths are specified with tight tolerances.

The waves reflected back from the sample arm 103 and the reference arm 102 interfere at the detector array 110. Since the interference signal is only created when the polarization in the reference arm 102 matches with that in the sample arm 103, in some embodiments, a polarization compensator 120 is used either in the reference arm or the sample arm. Polarization compensator 120 is also known as fiber optic polarization compensators. In some embodiments, the compensator comprises of 3 coils of fiber on 3 different paddles arranged in a series. The first fiber coil is a quarter wave plate, the second fiber coil is a half wave plate (typically the fiber is looped around twice for the same paddle diameter as the first paddle), the last fiber coil is a quarter wave plate. These 3 paddles can be rotated freely with respect to each other to produce any polarization state.

There is another type of polarization compensator, which applies pressure to the fiber to create birefringence. The slow axis is in the direction of the pressure applied. This fiber squeezer can be rotated around the fiber to rotate the direction of the slow axis. Thus, any arbitrary polarization can be created.

In some embodiments of the OCT systems, light exits a fiber tip in the reference arm and the light returns from a retro-reflecting mirror mounted in the air.

OCDR-OCT sub-system uses spectroscopic detection method. Basically the interferometric light exiting the detector arm 104 is dispersed via a grating. The spectra are acquired using a line-scan camera. The resulting spectra are typically (by way of example, not by limitation) transferred to a processor for inverse Fourier transforming and relevant signal processing (such as obtaining the complex envelope of the interferometric signal) for obtaining depth dependent (i.e., axial) reflectivity profiles (A-scans). The axial resolution is governed by the source coherence length, typically ˜3-10 μm. Two dimensional tomographic images (B-scans) are created from a sequence of axial reflectance profiles acquired while scanning the probe beam (by reflecting the probe-beam from a scanning mirror) laterally across the specimen or biological tissue.

A-scan: A-scan is a plot of reflectivity of scatterers and layers as a function of depth at a given lateral location. It is computed as follows:
a) The interferometric light exiting the detector arm is dispersed via a grating.
b) The dispersed light has a spectrum which is focused on a detector array or a line-scan camera. Thus, the grating unit disperses the partial returning light from the beam splitter and a dispersed light enters the detector array to produce a light spectrum.
c) The recorded spectra are typically transferred to a processor. The processor performs a data analysis using specific algorithms on the light spectrum.
d) An inverse Fourier transform of the spectrum is computed.
e) Relevant signal processing is performed (such as removing the duplicate data and strong spikes at the center of the inverse Fourier transform) using specific algorithms.
f) The resulting arrays are depth dependent (i.e., axial) reflectivity profiles (A-scans). Thus the system generates A-scans of the eye; if the eye is the specimen used in the system.
g) The axial resolution is governed by the source coherence length, typically ˜3-10 μm.

B-scan: Two dimensional tomographic images (B-scans) are created from a sequence of axial reflectance profiles acquired while scanning the probe beam laterally across the specimen or biological tissue. The following are detail steps:
An A-scan is acquired at a given lateral location.
A mirror is scanned using a scanner such as a galvanometer or a MEMS mirror in the optical delivery unit.
Multiple A-scans are acquired at various lateral locations.
A matrix is generated where columns indicate different lateral locations and rows indicate reflectivity at each depth in each A-scan.
The matrix is displayed as an image, which is also a B-scan.

In some embodiments of this invention, the grating disperses light and a lens focuses it into a detector array 110. By way of example, but not by limitation, this array can be a line-scan camera, which has quantum efficiency) at the operating wavelengths. The resulting data set is inverse Fourier transformed, processed in a processor 114 and displayed as a gray scale or pseudo-color image. By way of example, not by limitation, this processor can be a computer, off-the-shelf integrated circuit, application specific integrated circuit (ASIC), Field Programmable Gate Array (FPGA), a graphical processing unit (GPU) an embedded system or a microcontroller.

Extensions of the proposed interferometer: An interferometric 2D imaging system (Optical coherence tomography or OCT) can be constructed using the proposed interferometric system where the 2D images are obtained by laterally scanning the beam incident on the sample using a 1-D scanning mirror (which is a part of the optical delivery unit). An interferometric 3D imaging system can be constructed using the proposed interferometric system where the 3D data-sets are obtained by 2D laterally scanning the beam incident on the sample using a 2-D scanning mirror (which is a part of the optical delivery unit).

Both the 2D imaging systems and 3D imaging systems can be adapted for ophthalmic imaging by using a lens assembly (which is a part of the optical delivery unit) to focus the light on the retina.

In some embodiments, the optical delivery unit is integrated with the face-holder of the ophthalmic system.

An example lens assembly is described below (not as a limitation), but other lens assemblies could be used. The OCDR-OCT system can be adapted to measure retina by collimating the beam exiting the sample arm fiber, expanding the beam using a lens, shrinking the beam to project on the cornea, and the cornea and lens system of the eye will focus the beam on the retina.

In some embodiments, a fractional wave mirror is placed at the end of the reference arm of the OCDR/OCT/OFDR system. The fractional wave mirror comprises of a fiber-optic mirror preceded by a fractional [45 degrees (λ/8)] waveplate. Here λ indicates wavelength. The polarization of light incident on the wave plate is rotated by 45 degrees, and is directed to the fiber-optic mirror. The reflected light is further rotated by 45 degrees by the fractional [45 degrees (λ/8)] waveplate and hence the resulting polarization is orthogonal to the incident polarization. Polarization compensator 120 may not be necessary in this embodiment. A modified formula based on LeFevre is disclosed in this disclosure and which is as follows:

Mechanical stress on the fiber causes birefringence in the fiber. Stress can be generated by simply bending the fiber. According to LeFevre (U.S. Pat. No. 4,615,582), the fractional wave plate can be built by looping the fiber into N loops having a radius R. The refractive index difference Δn for two orthogonal polarizations is given by

$$Δ n = {b ( \frac{r}{R} )}^{2} \qquad ( Eq 2 )$$

b is a constant depending upon the photoelastic coefficient of the fiber, r is the radius of the fiber and R is the radius of the fiber loop. Thus, if we want to create a λ/m (where m is an integer) waveplate, which will introduce a path-length shift of λ/m between 2 polarizations, we'll need to create a loop of fiber length L to create the path-length shift of ΔnL. However, since the length of the fiber is also equal to 2πNR, where N is the number of loops, we get

$$( 2 π NR ) {b ( \frac{r}{R} )}^{2} = \frac{λ}{m} \qquad ( Eq 3 )$$

$$or$$

$$R = ( 2 π mN ) b \frac{{r}^{2}}{λ} \qquad ( Eq 4 )$$

To create a fractional wave plate of

$$\frac{λ}{8} ,$$

and N=1 (single loop), b=0.25, m=8, r=125 microns, λ=0.8 microns, we get

$$R = ( 2 π 8 ) 0.25 \frac{{( 125 )}^{2}}{0.8} = 5 π * 15625 = 24.54 cm \qquad ( Eq 5 )$$

Please note that a (2M+1)λ/m waveplate where M is an integer between −∞ to ∞ will have a similar effect as a λ/m waveplate. The corresponding equation is

$$R = ( 2 π mN ) b \frac{{r}^{2}}{λ ( 2 M + 1 )} \qquad ( Eq 6 )$$

Thus, if M=5 in the example above; R would be 2.23 cm, leading to a more compact loop. We could choose various values of M leading to an optimal design and size.

The waves reflected back from the sample arm 103 and the reference arm 402 interfere at the detector array 110. Since the interference signal is only created when the polarization in the reference arm 102 matches with that in the sample arm 103, in some embodiments, one can include by way of example but not by limitation a 45 degrees λ/8 waveplate in the sample arm 103 just before the light is incident on the optical delivery unit 108. Since the polarization of the retro-reflected light will be almost orthogonal to the incident light (considering the fact that the birefringence in the specimen 107 will modify the polarization state), the birefringence effects in the sample arm fiber 103 of the interferometer 400 will get cancelled. In an embodiment, the λ/8 waveplate is constructed using fiber optic components.

In another preferred embodiment, the λ/8 waveplate is a fractional-waveplate constructed using fiber optic components. It would be constructed in the optical delivery unit near the end of the fiber segment in the optical delivery unit. The fractional waveplate is located on the sample arm of the apparatus/system. It may be made an integral part of the optical delivery 108. The fractional wave mirror in the reference arm comprises of a fiber-optic mirror preceded by a fractional [45 degrees (λ/8)] waveplate. The polarization of the light incident on the waveplate is rotated by 45 degrees, and is directed to the mirror. The reflected light is further rotated by 45 degrees by the fractional [45 degrees (λ/8)] waveplate and hence the resulting polarization is orthogonal to the incident polarization. In another embodiment, a free-space-bulk 45 degrees (λ/8) wave plate is used at the end of the optical delivery unit. Polarization compensator 120 may not be necessary in these embodiments.

In some embodiments, the optical delivery unit 108 in the sample arm is integrated with the face-holder.

In another variation of this embodiment, the fiber optically integrated mirror can be replaced by a free space mirror 118. The light can be delivered to the mirror using optical delivery unit 108. FIG. 1 has standard free-space-mirror 118 in the reference arm, which still permits use of instant algorithms such as frequency resampling, dispersion compensation, and Doppler processing algorithms. In some embodiments, the system comprises of the optical delivery unit 108 in the sample arm.

In some other embodiments, the system is used for optical coherence tomography (OCT) imaging and the OCT system comprises of a tunable wavelength (or frequency) light source to implement swept-source OCT or optical frequency domain reflectrometry (OFDR) (as described in S R Chinn, E A Swanson, J G Fujimoto—Optics Letters, 1997; M A Choma, M V Sarunic, C Yan et al—Optics Express, 2003; Y Yasuno, VD Madjarova, S Makita, M Akiba et al—Optics Express 2005).

Frequency Domain OCT or swept source OCT or Optical Frequency Domain Reflectometry (OFDR): In some OCT sub-systems such as frequency domain OCT or swept source OCT or Optical Frequency Domain Reflectrometry (OFDR), the broad-band light source is replaced by a tunable frequency light source. The detector array is replaced by a single detector. The use of a grating is not needed for this embodiment. In this embodiment, a fiber-optically integrated mirror in the reference arm 102 of the OFDR-OCT system can be used. A tunable light source is chosen for light source 105 in this embodiment. The center wavelength most ideal for the retinal applications ranges from 750 nm till 1050 nm. The wavelength of the source is tuned very rapidly (e.g., at a rate of 10 kHz-10 MHz) within a spectral range of typically 10 to 100 nm around the center wavelength. The average power of such a source typically ranges from 0.1 mW to 20 mW depending upon the application. The source may be electrically operated. The existing commercially available sources operate on 110/220V 50/60 Hz power input. In future, these could be operated using lower voltages and battery operated while in transit. ADC (analog to digital converter) is added so that the electrical current is transformed.

In this embodiment there is no grating 113 and detector array. Instead a detector is added. It is a photo-diode (which produces an electrical current in response to incident light). The detectors for 300-1000 nm are typically made up of silicon. The detectors for 900-1700 nm are typically made up of InGaAs. These are high-speed detectors with typically 0 to a few hundred MHz bandwidth. In some embodiments more than one detector may be used to achieve dual-balanced detection. It is typically followed by a high-speed A/D (analog to digital) converter (ADC), e.g., 8-bit or 12-bit with a conversion rate of 1 to 20000 Mega Samples/second. The detector(s) direct(s) the signal to the ADC to generate a digitized signal. Typical responsivity of photodiodes is 0.1-1 mA/mW. The output voltages are typically −5 to 5V with typical 50Ω impedance. These assist in achieving typical line-rates (rate of acquisition of A-scans) of 10000 lines/s to 400,000 lines/s (can be higher than 10M lines/s in very high speed lasers). The digitized output of the A/D converter is typically directed to a computer or a processor using an Ethernet cable (e.g., Gigabit Ethernet) or a USB (typically 2.0 or 3.0) cable, or directly attached to the computer's PCI (Peripheral Controller Interface) bus etc. The processor generates A-scans and/or B-scans.

Since the interference signal is optimal when the polarization in the reference arm 102 matches with that in the sample arm 103, in some embodiments, a polarization compensator 120 is used either in the reference arm or the sample arm. polarization compensator 120 is also known as fiber optic polarization compensator. In some embodiments, the compensator comprises of 3 coils of fiber on 3 different paddles arranged in a series.

In some embodiments, a fractional wave mirror (as described earlier) is placed at the end of the reference arm of the OCT/OFDR system. The fractional wave mirror comprises of a fiber-optic mirror preceded by a fractional [45 degrees (λ/8)] waveplate. Here λ indicates wavelength. The polarization of light incident on the wave plate is rotated by 45 degrees, and is directed to the fiber-optic mirror. The reflected light is further rotated by 45 degrees by the fractional [45 degrees (λ/8)] waveplate and hence the resulting polarization is orthogonal to the incident polarization. Polarization compensator 120 may not be necessary in this embodiment.

The waves reflected back from the sample arm 103 and the reference arm 102 interfere at the detector array 110. Since the interference signal is only created when the polarization in the reference arm 102 matches with that in the sample arm 103, in some embodiments, one can include by way of example but not by limitation a 45 degrees λ/8 waveplate in the sample arm 103 just before the light is incident on the optical delivery unit 108 in the OCT/OFDR system. Since the polarization of the retro-reflected light will be almost orthogonal to the incident light (considering the fact that the birefringence in the specimen 107 will modify the polarization state), the birefringence effects in the sample arm fiber 103 of the interferometer 100 will get cancelled. In an embodiment, the λ/8 waveplate is constructed using fiber optic components. Polarization compensator 120 may not be necessary in this embodiment.

In another preferred embodiment, the λ/8 waveplate is a fractional-waveplate constructed using fiber optic components in the OCT/OFDR system. It would be constructed in the optical delivery unit near the end of the fiber segment in the optical delivery unit. The fractional waveplate is located on the sample arm of the apparatus/system. It may be made an integral part of the optical delivery 108. The fractional wave mirror in the reference arm comprises of a fiber-optic mirror preceded by a fractional [45 degrees (λ/8)] waveplate. The polarization of the light incident on the waveplate is rotated by 45 degrees, and is directed to the mirror. The reflected light is further rotated by 45 degrees by the fractional [45 degrees (λ/8)] waveplate and hence the resulting polarization is orthogonal to the incident polarization. In another embodiment, a free-space-bulk 45 degrees (λ/8) wave plate is used at the end of the optical delivery unit. Polarization compensator 120 may not be necessary in this embodiment. The face holder comprises of the optical delivery unit 108 in some embodiments.

In the OFDR-OCT system fiber optically integrated mirror can be replaced by a free space mirror 118. The light can be optionally focused on the mirror using an optical delivery unit 108. The optical delivery unit 108 can be fixed to the face holder in some embodiments.

In another embodiment, the OFDR-OCT system can be used without the fiber stretcher. The mirror 118 in the reference arm is able to move back and forth to match with the pathlength in the sample arm. The mirror motion can be achieved by a translation stage or a motorized stage or a galvanometer or a scanner. The face holder comprises of the optical delivery unit 108 in some embodiments.

Extensions of the proposed interferometer: An OFDR/OCT interferometric 2D imaging system can be constructed using the proposed interferometric system where the 2D images are obtained by laterally scanning the beam incident on the sample using a 1-D scanning mirror (which is a part of the optical delivery unit). An interferometric 3D imaging system can be constructed using the proposed interferometric system where the 3D data-sets are obtained by 2D laterally scanning the beam incident on the sample using a 2-D scanning mirror (which is a part of the optical delivery unit).

Bulk of the components of the OCT/OFDR system can be placed in the base of the ophthalmic system in some embodiments. The optical delivery unit in the sample arm can focus light on the eye. The optical delivery unit can be attached to the face holder in some embodiments.

In some embodiments, the OCT/OCDR/OFDR/SLO apparatus/system operates on batteries. In some other embodiments, these batteries can be rechargeable batteries. The batteries can be charged independently or by connecting a charger to the apparatus. The charger can source power from the electrical wiring in a building. The charger (termed a vehicle charger) can also source power from a vehicle such as a car or a bus or a truck or a van. The charger can also source power from the vehicle's engine.

### DESCRIPTION OF THE PREFERRED EMBODIMENT

The system can be used to image mice, rats, other small animals, zebra-fish, tadpoles, mid-sized and large animals, primates, dogs, and humans. This system could also be used to image non-living specimens, test objects, test materials, excised tissues from animals and humans.

The system comprises of a positioner to house the specimen. In some embodiments, the specimen can be an animal, or fish holder or a non-living target. The animal positioner can be modified to mount mice, rats, squirrels, other small animals, zebra-fish, tadpoles, mid-sized and large animals.

This system can be used to optionally combine OCT (optical coherence tomography), fluorescence imaging (and/or SLO, scanning laser ophthalmoscope), OCT-angiography, Doppler OCT, adaptive optics OCT and/or adaptive optics SLO and/or adaptive optics Doppler OCT), and/or adaptive optics OCT-angiography.

In one embodiment, these modes can utilize non-overlapping wavelength bands. For example, SLO and OCT can be combined by allocating a short wavelength range for fluorescence excitation and SLO reflectance signals, an intermediate wavelength range for fluorescence SLO detection, and a higher wavelength range for OCT. These bands might typically involve the visible excitation and emission bands of a fluorophore of interest (e.g. GFP), and NIR for OCT.

Alternatively, these bands can be divided. For example, the system might use a broadband visible light source and a fluorescence filter cube (FFC) engineered for use with multiple, non-overlapping excitation and emission bands to detect fluorescence from multiple fluoroscopes in a single scan.

A fluorescence filter cube comprises of an excitation filter, an emission filter and a dichroic beam-splitter. The excitation filter could be a band-pass or short-pass filter permitting the selection of fluorescence excitation light (either via transmission or reflection of the incident light) incident on the target.

The emission filter could be a band-pass or long-pass filter permitting the selection of fluorescence emission light (either via transmission or reflection of the light emitted from the fluorescing target).

A dichroic beam-splitter in FFC permits separation of the fluorescence emission and excitation light paths. For example, it may reflect the excitation light and transmit the fluorescence emission light wavelengths.

An FFC could permit only one fluorescence band or multiple fluorescence bands.

The system could be equipped with multiple fluorescence channels. Alternatively, with a single fluorescence channel, each fluorophore can be individually scanned by adjusting the excitation filter at the light source to selectively excite individual fluorophores. Alternatively, the wavelength bands can be allocated in other ways.

In one embodiment (called split-spectrum mode), a shorter NIR band can be utilized for OCT as well as excitation for infrared fluorophores, with a longer-wavelength band allocated for fluorescence SLO detection.

### System Description

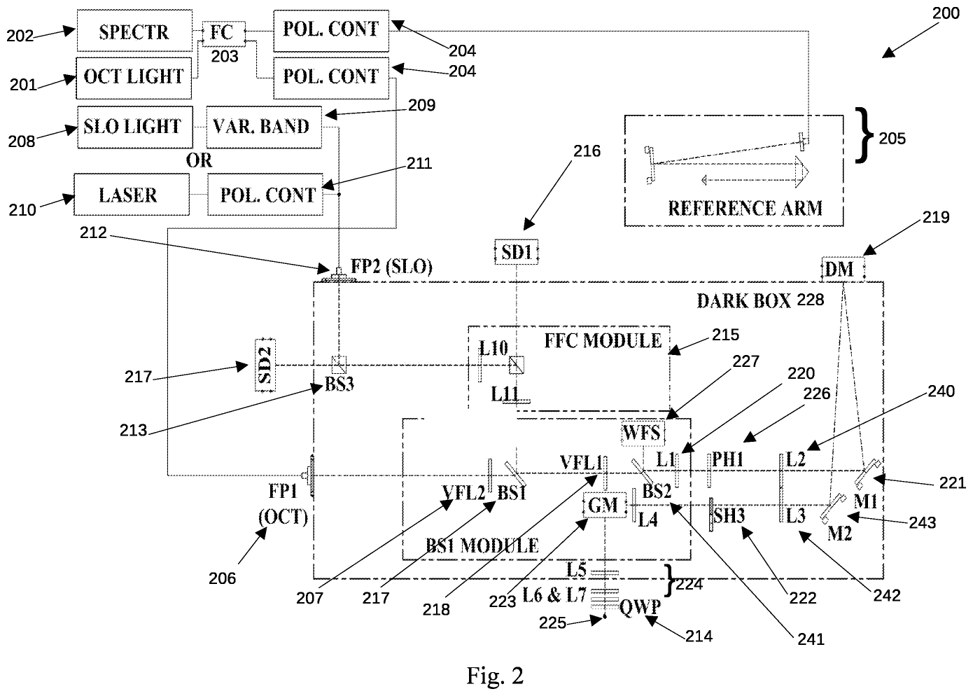

The system (FIG. 2) comprises of an OCT subsystem, the SLO subsystem, and optics common to both.

The OCT subsystem comprises a broadband OCT light source 201, such as a single superluminescent diode or an array of superluminescent diodes, and a spectrometer 202. In an embodiment, these are joined via a fiber coupler 203 to a pair of polarization controllers 204 and, to the OCT reference and sample arms. These polarization controllers 204 can be mounted so that they can be accessed and adjusted by the user during system operation. The reference arm 205 reflects the portion of the OCT excitation directed to it by the fiber coupler back to generate interference of the light. In embodiment, this arm includes a motorized or translating stage that can be used to set the distance from which the light is reflected back (reference arm path-length). The sample arm connects to the SLO/OCT-common-optics through an adjustable fiber port 206.

Additional glass or material of various types, to balance the optical dispersion of the reference and sample arms, can be added to the reference arm, the sample arm, or both. (Not shown.) Optical dispersion is matched in hardware by using at least 2 types of materials with different second and third order dispersion coefficients. For example, highly dispersive material such as SF11 and a moderate dispersive material such as BK7 could be used to match the dispersion in both the reference and sample arms.

An electrically-controlled-variable focus lens 207 (VFL2) adjusts the OCT sample arm light to compensate for chromatic aberrations due to wavelength differences between the OCT and SLO light. This variable focus lens can be controlled manually or automatically.

As some implementations of the system will operate on variable wavelengths, the chromatic aberrations can be expected to vary as a result.

The SLO subsystem is comprised of excitation, filtering, and detection. SLO excitation can be from a broadband (in some embodiments, the light can be visible, infrared or ultraviolet) light source 208, filtered through a variable bandpass filter 209.

In an alternate embodiment, SLO excitation can be provided by a fixed-wavelength laser 210 such as a 488 nm cyan laser, and an optional polarization controller 211. This light connects to the SLO/OCT common optics through an adjustable fiber port 212. It is then divided by a beam splitter 213. This beam splitter might reflect a certain ratio of the light, e.g. reflecting 70% and transmitting 30%. Alternatively, it might reflect or transmit light based on its polarity. This option might utilize a polarization controller either at the laser 211, or between the fiber port 212 and the beam splitter 213.

In one embodiment, a quarter wavelength plate 214 can be used to rotate the polarization of the light backscattered from the subject. This quarter wavelength plate 214 can be mounted to permit rotation by the user for adjustment through a machined crown, secured by a plastic-tipped setscrew or thumbscrew, or a rotational optical mount 503. The beam splitter 213 redirects the SLO excitation towards the fluorescence filter cube FFC module 215.

In one embodiment, the FFC module 215 uses a translating (optionally motorized) stage 301 to change between multiple fluorescence filter cubes 302. The FFC cubes typically contain an excitation filter, a dichroic, and an emission filter. Each cube would have filters selected for a specific fluorophore (i.e., fluorescence band) or sets of fluorophores.

In an embodiment, the motorized or translating FFC stage 301 positions the FFC cubes 302 so that the SLO excitation passes through the excitation filter and reflects off of the dichroic (such a dichroic is called longpass-dichroic-beamsplitter). The fluorescence filter cube module 215 may have lenses that focus the light onto the filters.

The light returning from the specimen again passes through the fluorescence filter cube module 215. Fluorescence passes through the long-pass-dichroic beamsplitter and emission filter in the fluorescence filter cube 302. This light will then be detected by the fluorescence detector 216, which might be a photo-multiplier tube. In turn, light reflected by the subject will be again reflected by the dichroic in the fluorescence filter cube 302, towards the reflectance detector 217, which might be an avalanche photodetector or a photomultiplier tube (PMT).

In some embodiments, a shortpass-dichroic-beamsplitter which would transmit the excitation light and reflect fluorescence emission light could be used.

In some embodiments, these detectors may have lenses to focus the light upon them, and shutters to block light during service. These shutters might be mechanically controlled, for example, to prevent exposure of sensitive optical elements to light when the system is powered down and the cover removed for service.

In some embodiments detectors may be fully or partially con-focal, proceeded b a focusing element and pinhole, with size equal to or larger than the diffraction limit.

The excitation from OCT and SLO subsystems are combined by a beam splitter 217 in the integrated beam splitter module 501. This beam splitter 217 can be a single element.

In an embodiment, this beam splitter 217 can be selected from an array of optical elements installed on a translating or motorized stage 502 that permits various choices of 217 to be selected from a longwave pass dichroic, a shortwave pass dichroic, or a mirror. The various choices for 217 would permit various imaging modes. A longwave pass dichroic will pass the longer wavelength band allocated to the OCT (e.g. NIR) and reflect shorter wavelength band allocated to the SLO (e.g. visible light). This mode permits the combined operation of both SLO and OCT (This is the commonly used imaging mode).

In another embodiment, the shortwave pass dichroic 217 permits OCT to operate in a shorter wavelength band, with the fluorescence due to the OCT excitation occurring in an allocated longer wavelength band. This mode is called split-spectrum mode. For example, it might be used with fluorophores such as ICG (Indocyanine Green), allocating one shorter wavelength NIR band for OCT and fluorescence excitation, and one longer wavelength NIR band for SLO fluorescence detection.

In an embodiment, a mirror can be chosen for element 217 to permit SLO operation over wavelength ranges overlapping with the OCT source light. This would be an SLO only imaging mode.

The combined OCT and SLO excitation, depending on the mode, pass through an electrically-controlled variable focus lens 218. This VFL can be used to electronically make small adjustments in the focal length of the system optics. It can also be utilized to automate the optimization of focal length, and reference arm length and the working distance (the distance between the objective and the subject) in the sample arm. This combined beam then is directed to a (typically electrically) deformable mirror 219.

In an alternate embodiment, the VFL1 218 could be placed in a path exclusive to SLO (anywhere in the light-path-between element 217 BS1 and the fiber port FP2). 207 (VFL2) can be used to focus the OCT sample arm light at a depth location of interest. That way the SLO and OCT can be focused anywhere independently per the operator's choice. In an embodiment, the operator could use VFL 218 to focus the SLO image in the same focal plane as the focused OCT en-face image. Thus chromatic aberrations (difference in focal length at different wavelengths of light used for OCT and SLO) could be used by independently controlling the SLO and OCT images using independent VFLs.

In an embodiment, the beam out of the VFL 218 passes through a telescope comprised of two achromatic lenses 220 and 240 to expand the beam as it and is directed towards the deformable mirror 219 by a beam-folding mirror 221. The beam is then directed by a second folding mirror 243 and reduced by a telescope comprised of two achromatic lenses 241 and 222.

This deformable mirror 219 permits adaptive optics by changing shape to compensate for optical aberrations in the subject and elsewhere in the system. These deformable mirrors can be paired with adapters so that they can be interchanged without major modification or realignment to the system. In another embodiment, a flat mirror could be substituted for a less expensive system without adaptive optics.

An optional emergency shutter 222 blocks excitation in cases of system failure or in response to an emergency stop switch. In an embodiment, this shutter has a rapid electrical control, and might monitor the galvo control signal, so as to prevent a localized area of the eye from being exposed to high-power light for too long if the galvos stop.

This beam is then reflected by single or dual axis scanning mirrors 223. Many scan patterns are possible for example, a raster pattern, a line scan, an XY scan, a circle scan, radial line scans, etc. An objective 224 then causes the beam to converge toward a moving spot on the subject 225. OCT and SLO reflectance, and SLO fluorescence, will follow the optical path back. A pinhole 226 blocks stray light.

The deformable mirror 219 can be adjusted using software programs to try to optimize the resulting images.

Alternatively, some of the returning light can be diverted by a beam splitter to a wavefront sensor 227 to perform sensor-based adaptive optics.

In an embodiment, the common optics of the system are housed in a dark box 228 to eliminate stray light. To permit airflow while limiting light intrusion, this dark box 228 can be equipped with dark vents 601 that have curved internal passages that permit some airflow while blocking direct light leakage. These dark vents 601 can share mounting features with modular commercial cable pass-throughs 602, so that either cable routing or airflow routing could be configured without needing to modify the dark box walls.

### Variable Filters Module

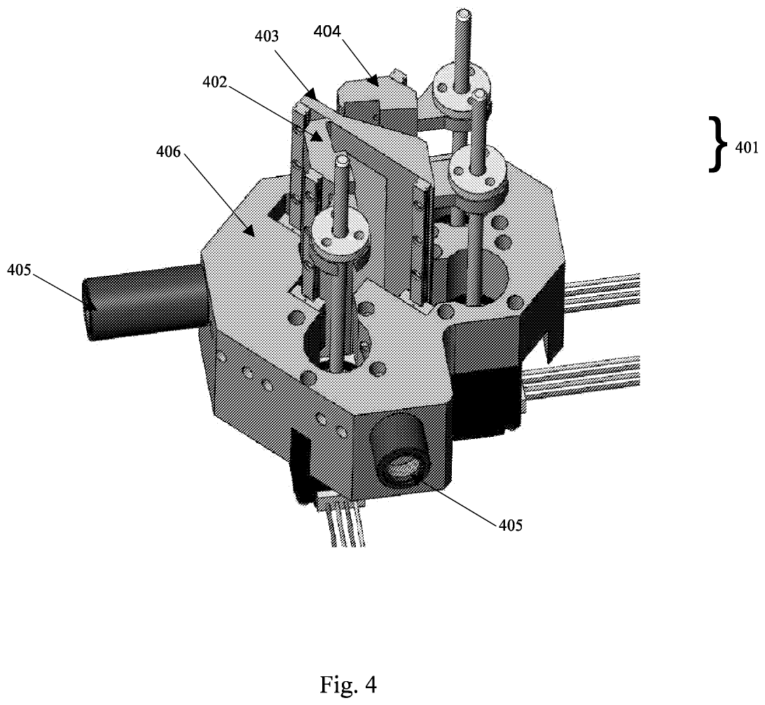

In an alternate embodiment, the fluorescent filter cube stage 215 might be replaced with a set of linear variable filters and dichroic beam-splitters 401 (FIG. 4). In an embodiment, the spectral properties (e.g., cut-off wavelengths, cut-on wavelengths, etc.) vary with distance (or position) on the filter.

In an embodiment, the two modules can be engineered to have common mounting features so as to be interchangeable. These linear variable filters would permit a continuous color (i.e., wavelength) range of fluorescence and reflectance excitation and detection.

A number of linear variable filters and dichroic beam-splitters can be clustered together, analogous to the discrete filters and dichroic beam-splitters of a fluorescence filter cube. Analogous to the filters of the fluorescence filter cubes, this module would have an excitation filter 402, a dichroic 403, and one or more emission filters 404.

In an embodiment, the cut-off wavelengths for some or all of these filters might vary nonlinearly as a function of location on the filter or translation of the filter. For example, they might vary exponentially.

These could be driven by a single motor. Alternatively, there might be one motorized stage for each filter type, for example, to allow the adjustment of the excitation and emission filters independently.

In an embodiment, lenses 405 focus the incoming beams to have a focal point near the filters. As the cutoffs of the filters vary along their length, a smaller spot will have a sharper cutoff. This focus can be aided by having multiple sets of lenses, each focusing on a specific filter.

In another embodiment, the filters can be placed physically close together, near a common focal point of a set of lenses.

These linear variable filters can be cascaded to give a sharper transmission cutoff than an individual filter could offer. The cascaded filters can be fabricated with matched curves, and aligned to each other with a static offset. Furthermore, cascaded filters can be mounted on separate motorized stages. The positioning of such cascaded filters would need to be carefully adjusted. Again, the wavelength cutoffs of the linear variable filters vary along the length of these filters. The sharper cutoff from cascaded filters requires both filters to be positioned so that they cut off equally. If the cutoff is driven by one filter or the other, the combined cutoff won't be sharper than the cutoffs of the individual filters.

In this embodiment, stray light would need to be blocked, such as by mounting the optics in a block 406 that covered most paths for leakage light. Conventional filters transmit or block light uniformly across their surface. In contrast, the transmission of light varies along the length of linear variable filters. Light that should be blocked could leak through due to being offset towards one end of the filter or the other. If mounted to separate motors, the filters and dichroic beam-splitters could be inverted relative to each other. For example, the dichroic could be mounted so that the 50% transmission wavelength decreased top to bottom, while the emission filter could be mounted so that the 50% transmission wavelength increases top to bottom. This limits leakage from getting past both filters by being either high or low. Alternatively, the filters can be non-inverted, but mounted on alternate sides of a focal point.

Thus the instant invention could permit a researcher choose an arbitrary fluorophore with an excitation wavelength within a specified band and perform fluorescence detection.

In an embodiment, the light source is divided into at least 2 separate paths. One path would illuminate the OCT interferometer system and the second path would illuminate the SLO system. These two paths would have appropriate potentially variable filters to control the spectra of light entering into each imaging modality sub-system. So the SLO part could have filters with bandwidth appropriate for the fluorophore. The OCT path would have a filter with the bandwidth that would give an optimal resolution and a center wavelength that would permit deep penetration within a given specimen.

This multi-modality system would minimize image-registration errors and also speed up the pace of the imaging experiments and the research.

### Adaptive Optics

In an embodiment, the deformable mirror can be manually adjusted to correct the wavefront. This would give better resolution OCT and SLO images.

In one embodiment, OCT image can be optimized using adaptive optics. The SLO images would be automatically optimized. If the OCT and SLO lights have different wavelengths, the difference in focal position could be corrected by setting both VFLs (VFL1 and VFL2) correctly.

In an embodiment, VFL1 sets the focus of the SLO beam to get an optimal SLO image. VFL2 is set to get an optimal OCT image by compensating for chromatic aberrations. The deformable mirror is set to an optimal shape so that the highest resolution is obtained for OCT and SLO images.

In an embodiment, the optimal shape of the deformable mirror is obtained by analyzing reflections from a certain depth-layer within 1-D, 2-D or 3-D OCT dataset.

In another embodiment, the figures of merits (FoM) are computed for various shapes of the deformable mirror, and the shape with an optimal figure of merit is computed and set.

In an embodiment, the FoMs are computed for various amplitudes of Zernike polynomials based mirror shapes, and an optimal shape is selected based on the FoMs. This is called Modal optimization.

In an embodiment, the FoMs are computed for various amplitudes of the zones in the mirror, and an optimal shape is selected based on the FoMs. This is called Zonal optimization.

In an embodiment, the optimal images are obtained by sensing the wavefront from the WFS 227 and correcting the wavefront error by controlling the deformable mirror using a signal from the wavefront sensor. The difference in focal position could be corrected by setting both VFLs (VFL1 and VFL2) correctly.

In one embodiment, the shape of a deformable mirror present in the imaging path can be adjusted. Adjustment can be either through coefficients of a suitable basis set (for example, Zernike polynomials) from which actuator positions are derived, or direct control of said actuator positions (sometimes termed zonal or pixelized control). Adjustments to the mirror shape serve to compensate for optical aberrations in the system and eye, so as to reduce the size of the spot formed on the retina, thereby increasing imaging resolution.

Adjustments may be either:
Manual in nature, made by the operator, based on subjective observation of image quality.
Automatic in nature, made by an optimization algorithm designed to improve a specified figure of merit (FOM) calculated from an image or profile. Said image or profile can be derived from depth-selective OCT EnFace reconstruction, OCT-angiogram, fluorescence or reflectance measurements. Available figures of merit may include but are not limited to: integrated intensity (e.g., the average of all the pixel intensities or average of square of pixels intensity), contrast (e.g., the ratio of the image standard deviation and image mean or the difference in the highest and lowest pixel values, etc.), sharpness (e.g., the average of the magnitude of the gradient values at each pixel location or the variance of the pixels in each image) or vesselness. Application of this technique may be jointly termed an adaptive optics optimization implementation.
Automatic in nature, made by an optimization algorithm to improve wavefront flatness, as measured by a wavefront sensor (WFS 227) placed in the returning beam path.

In one embodiment, two variable focus lenses allow for compensation for the different operating wavelengths of the SLO and OCT beams, thereby allowing both modalities to be brought into focus and corrected simultaneously using the deformable mirror, even in the presence of chromatic aberrations from the system and subject under test. This may be termed synchronized multimodal focusing. This may be implemented with variable focus lenses either configured;
1. Wherein one variable focus lens (VFL1) adjusts collimation independently for the SLO beam and another (VFL2) adjusts collimation collectively for both OCT and SLO beams.
2. Wherein one variable focus lens (VFL1) adjusts collimation independently for the SLO beam and another (VFL2) adjusts collimation independently for the OCT beam.

In one embodiment, adjustment of one or more variable focus lenses is integrated into an adaptive optics optimization implementation such that variable focus lenses provide initial gross adjustment to focusing, extending the dynamic range of available wavefront correction.

In one embodiment, synchronized multimodal focusing is used to allow the operator to identify the depth of a structure within the volume of a specimen using OCT, and then infer the characteristics at the depth of the said structure when observed in another modality, such as fluorescence imaging.

### OCT-Angiography

In one embodiment, multiple OCT volumetric data sets are acquired sequentially from the same region of a subject. These are used as input to an algorithm for identification of vessels through variation in the recorded intensity, brought about through the influence of probe-beam laser speckle on mobile scattering centers within the flowing fluid. This technique is termed OCT-angiography. Data may be assembled either through:
1. Repetition of each B-scan, with data acquired on either forward, backward, or both scan directions.
2. Repetition of multiple complete OCT raster images

In one embodiment, the measurement of flow rate at a given point in the image or profile is made by the statistical method of variance calculation (or similar statistical technique) which is calculated from the integrated intensities within a selected OCT depth range of interest.

In one embodiment, the measurement of flow rate at a given point in the image or profile is made by the statistical method of variance calculation (or similar statistical technique) which is calculated independently for each voxel within a selected OCT depth range of interest.

In one embodiment, the sequential scans are first aligned before calculation of variance using the method of auto-correlation or similar. This provides suppression of large scale motion artifacts inherent to observation of a living subject.

In an embodiment multiple OCT volumes are required from the same region. The variation in OCT intensity is used to identify blood vessels within these volumes. This provides OCT-angiography data.

In another embodiment, multiple B-scans are acquired at the same location. The variation in the intensity of light backscattered from blood vessels would be higher as opposed to the variation in the intensity of light backscattered from regular tissues.

In an embodiment, adaptive optics can be used to improve the resolution of OCT-angiography.

### OCT Spectroscopy

In one embodiment, depth-resolved back-scattered spectroscopy could be performed by analyzing the OCT interferometric data at different depths.

### Mouse Positioner

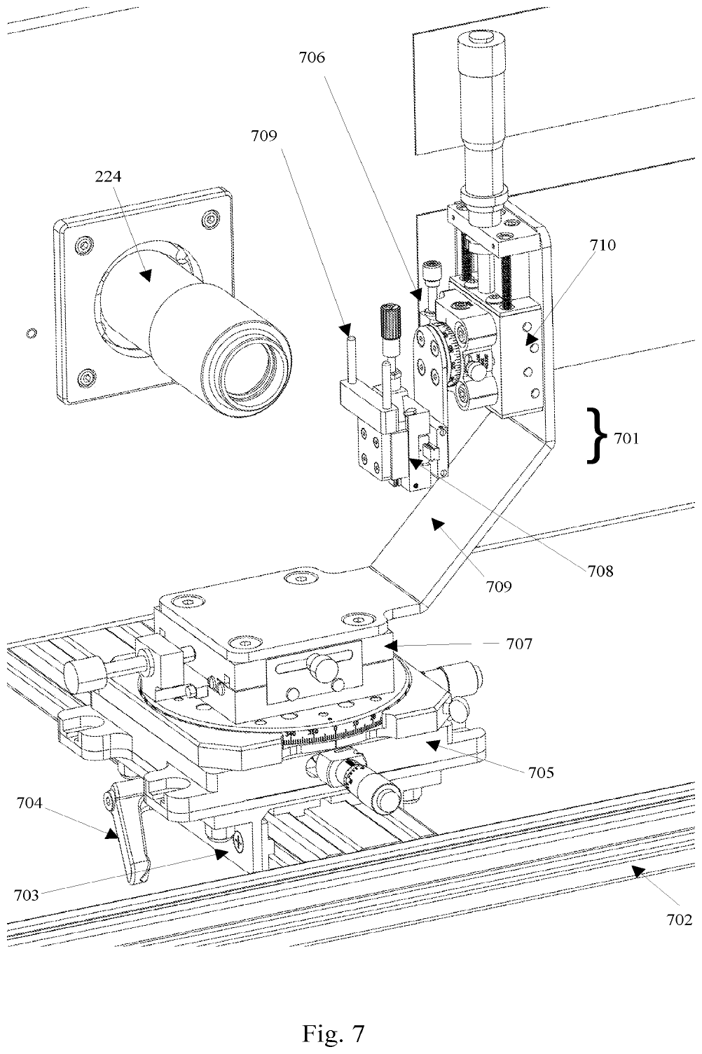

In an embodiment, an animal positioner can be used to mount an animal and align its eye with the system for imaging. In another embodiment, the animal positioner could be a mouse positioner (FIG. 7).

In an embodiment such an animal positioner could be used to perform tests and measurements on the animal using modalities other than OCT and SLO.

In another embodiment, the animal positioner could be used for a core system performing tests, measurements or imaging using OCDR and/or OCT and/or OFDR and/or OCT angiography, and/or Doppler OCT and/or spectroscopy and/or OCT spectroscopy and/or adaptive optics and/or fluorescence and/or reflectance, and/or SLO technologies.

The positioner could also be used with core systems performing therapy and/or optical surgery and/or laser surgery, and/or other electrical and/or chemical and/or mechanical surgeries,

The positioner could also be used for making electroretinogram measurements of mice and other animals.

A mouse positioner 701 supports the primary functionality of the core system by positioning and stabilizing the mouse.

In an embodiment, a conceptually equivalent positioner could be applied to other small animals such as rats, shrews, etc. Many aspects of the positioner described below could also be applied to other small animals, such as fish, etc. This positioner can be rigidly connected to the main system by a structural subframe 702 or by mounting both to a table such as an optical table. In the embodiment shown, the subframe 702 is constructed of slotted aluminum extrusion.

In an embodiment, the positioner 701 provides for the rapid engagement and disengagement through a linear slide 703 that is secured by a brake lever 704. A travel stop provides a repeatable engaged position.

Conventionally, the Z axis is along the objective 224. The horizontal axis is X, and the axis perpendicular to X and Z (vertical axis) is Y. In an embodiment, the positioner 701 also provides for the rotation of the subject by a first stage 705 that provides rotation around the Y axis and a second stage 706 that provides rotation around the X axis. This X axis rotation stage provides fine adjustment, and a fine adjustment lock, and a general rotation lock. In another embodiment, the first rotation stage 705 also provides for translation of the positioner along X and Z, to center the positioner 701 relative to the objective 224. In another embodiment, separate X and/or Z translating stages are used. In one more embodiment, another linear translation stage 710 provides centering along Y. Together, the X, Y, and Z translation provides for the proper alignment (or centering) of the rotational axes relative to the objective 224.

If the rotational axes were not centered relative to the objective, rotating the subject would also result in the translation of the subject, which may be undesired. This is the key advantage of the proposed system and method,

In an embodiment an additional set of translations is useful to align the imaging subject relative to the objective 224. These translations are referred to as X′, Y′, and Z′. A stage 707 near the base provides for X′ translation. An optional stage 708 near the mounting pins 709 provides for both Y′ and Z′ translation. In another embodiment, Y′ and Z′ could be separate translators.

XYZ are on a static reference frame (for the positioner) while X′Y′Z′ are on a rotating reference frame (for the animal housed on the positioner).

In embodiment, structurally this can be accomplished with only a single L bracket 709. This is because the X translation stage 707 could be moved below the parallel Y rotation stage 706, and the Y translation stage 710 could be moved above the parallel X rotation stage 705. In another embodiment, three angle brackets could be used to setup the rotating frame of reference.

In an embodiment, the X′, Y′, and Z′ linear translation stages are omitted. This positioner would still have linear translation in X, Y, and Z that could be used to align the subject to the objective. It would also have two rotational axes. However, as the rotational axes wouldn't generally be centered to the objective, the subject would translate with respect to the objective as it is rotated, and would need to be realigned using the translation stages.

### Cartridge

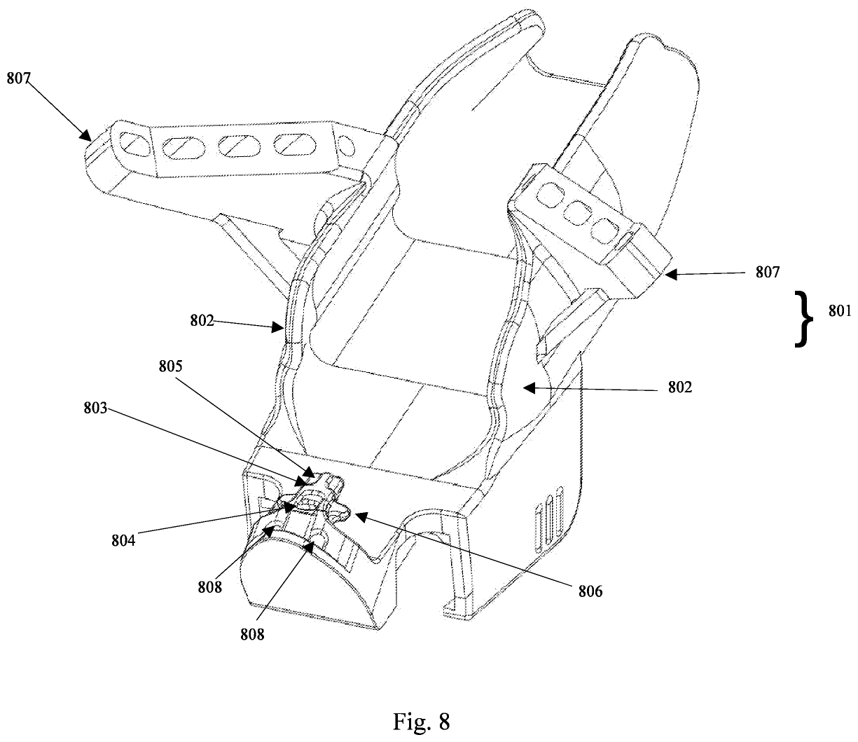

In an embodiment, to facilitate rapid and repeatable placement of the mouse, a cartridge 801 is used (FIG. 8). The cartridge 801 is optionally angled so that the mouse eye axis is approximately aligned with the objective 224 axis. Additionally, in another embodiment, this angling also aligns the mouse's nasal/temporal axis with the Y rotation stage 705 axis, and the mouse's superior/inferior axis with the X rotation stage 706 axis. This results in anatomically meaningful rotation. That is, operators wishing to rotate the mouse to change the nasal/temporal or superior/inferior angle would be able to make the rotation directly, without needing to approximate the desired rotation using multiple rotational axes that are not relevant to the mouse's anatomy.

As the cartridge 801 will be angled in use, it has side walls 802 to support the animal's torso. This cartridge can be fabricated in different sizes, and side bolsters could be added if necessary. In an embodiment, the cartridge can also provide for other means of securing the animal (or mouse). These other means might include velcro straps, surgical tape, etc.

In an embodiment, to maximize the repeatability of the placement of the mouse's eye, the cartridge can have an integrated bite bar 1402 or palate bar 803. A bite bar 1402 locates the mouse's head by hooking the mouse's incisors over a bar. A palate bar 803 locates the mouse's head by engaging the mouse's incisors in a hole 804, and also engages the mouse's molars on steps 805. Side features 806 can be used to depress the mouse's jaw, so that the mouse can be placed on the palate bar 803.

In an embodiment of the invention, for ambidextrous use, the cartridge can have a pair of mounting arms 807. These would slide over the mounting pins 709 in a quasi-kinematic fashion so as to locate the cartridge in a deterministically repeatable position. In the embodiment of FIG. 8, these arms have a base surface, a deep hole, and a short slot. Each would be configured to position one eye for imaging. A mounting scheme that is intended to precisely position the subject's eye, as opposed to the subject in general, is highly advantageous because it permits one subject to be removed and replaced, or another subject to be placed, without having to repeat coarse alignment. Time is generally limited, so being able to go directly to final alignment of the subject is highly beneficial.

Technically, a kinematic coupling is one that constraints all six degrees of freedom of an object, but does not overconstrain them. An embodiment with a fully kinematic coupling is possible, but would lack the mechanical simplicity and economy of the embodiment shown in FIG. 8. This embodiment, with a hole, short slot, and mating surface, precisely and deterministically locates the cartridge, but is not technically kinematic. The mating surface engages last, to effectively constrain one degree of freedom, while in theory, it would try to constrain three.

In another embodiment of a cartridge 1601, the mounting arm is configured with the short slot 1602 is near the top of the pin 1603. This embodiment might be advantageous as it can reduce the distance over which the cartridge can snag on the pins. It also separates the steps of aligning the cartridge hole 1604 on a pin, and then aligning the cartridge short slot 1602 on a pin. In the embodiment of FIG. 8, these steps were concurrent.

In an embodiment, to switch to imaging the other eye, users would first lift the cartridge 801 off the mounting pins 709. Then they would rotate the positioner 180 degrees using the Y axis stage 705. This would, for example, move the positioner 701 from the right of the objective 224 to the left. The user would then mount the cartridge 801 by sliding the other mounting arm 807 over the mounting pins 709. In mice and similar animals, the globe of the eye can be popped slightly out of the orbit, i.e. proptose, without injury. For mice and similar animals, this cartridge 801 can be engineered for either the proptose or non-proptose eyeball position.

In another embodiment, two cartridges could be produced, each with one arm configured to position one of the mouse's eyes. Such single sided cartridges 1401 might have support on the one downward side and have the other side open for easy insertion and removal of the mouse. To switch from one eye to the other, users would need to move the mouse from e.g. a right-eye cartridge to a left-eye cartridge, in addition to rotating the positioner.

This cartridge 801 might be 3D printed to provide for a wide range of sizes and configurations, and to integrate a number of features and channels that would be difficult to produce by conventional techniques such as machining or injection molding.

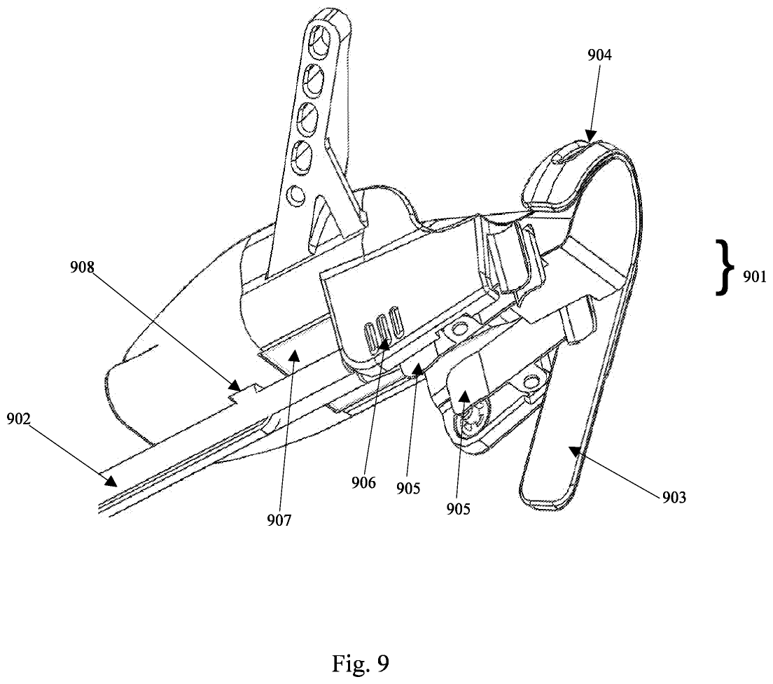

In an embodiment (FIG. 9), to support the use of gas anesthesia systems, the cartridge assembly 901 can have an integrated gas manifold with channels traveling to symmetric inlet and outlet 808 near the mouse's nose. Alternatively, the inlet and outlet might be asymmetric, for example, having a dual inlet 1502 near the mouse's jaw, and a single outlet 1501 near the nose, to provide flow over the mouse's mouth as well. The dual inlets might connect to a single machined passage 1503. This can be useful in case the angle of the mouse causes an exception to a mouse's generally-nasal respiration. These vents 808 or 1501 and 1502 will be connected via the manifold to tubes 902 that can connect to the cartridge, e.g., to barb fittings. These configurations of the gas anesthesia manifold are beneficial in that they will translate and rotate with the mouse, and won't excessively block access to the mouse's eye.

In an embodiment, the mouse's muzzle can be secured down to the cartridge using a retainer 903 or a flexible rubber strap. Such retainers could have a slot 904 to permit the mouse to freely breathe room air, or be without such a slot, to partially enclose the mouse's nose to contain anesthesia gases. Similarly, rubber straps can be narrow or wide to permit free breathing or the containment of anesthesia gases, respectively. The retainer 903 or strap will be secured by one or two spring clips 905 that can be opened by squeezing from the inside against the outside wall. Ridges 906 give an indication of where to squeeze that is visible from the sides or above. In another embodiment, the straps could be secured by two thumbscrews 1502. In yet another embodiment, the mouse's muzzle can be secured down by an elastic strap that routes around horizontal pins 1403 and through vertical pins 1404 to be secured by cordlocks 1405 that hook into the cartridge. This configuration is advantageous as the elastic strap can be routed and secured with one hand if needed.

In another embodiment, additionally, the cartridge assembly 901 can have an integrated resistance heater 907 and temperature sensor that can rotate with the mouse. This sensor can be used to control the heater temperature if the mouse's body temperature is not measured directly. A cable connects the cartridge to a controller. This configuration of the heater is beneficial in that it will translate and rotate with the mouse. The cartridge assembly 901 can have a rear anchor point 908 to provide strain relief for the tubes and cable.

A key benefit of this cartridge concept for ophthalmological use is that it specifically locates individual eyes in a highly repeatable manner, as opposed to locating the subject's body and requiring the operator to rotate and translate to bring an eye into position. This benefit might be extended to other body parts with a suitably modified cartridge.

An additional benefit is that any adjustment to the eye position is implemented by moving the cartridge, as opposed to moving one body part relative to the rest of the body. For precise adjustment, a high mechanical advantage e.g. by a fine-pitched adjustment screw, is needed, which will give poor feedback on the actual forces applied to the subject.

A further benefit is that the cartridges can be supplied in specialized sized and shapes, reducing the need to side bolsters or other adjustments that might be time-consuming, and that might increase the variability in the positioning of the subject's eye.

A stand can be provided to hold the mouse cartridge upright for mouse insertion into the cartridge assembly 901 and preparation. These steps are easier when the subject is level. Once prepared, the cartridge and mouse can be transferred from the cartridge stand to the positioner 701. Additionally, the separable stand permits a next subject to be prepared while a current subject is being imaged. A plurality of stands and cartridges could be used in this manner to facilitate the concurrent preparation of many test subjects, to be rapidly switched onto the positioner for imaging.

### Centering and Alignment

The cartridge assembly 901 and the positioner 701 are engineered to give highly repeatable placement of the mouse's (or animal's) eye. Some tools are helpful for the initial centering of the positioner and alignment of the mouse.

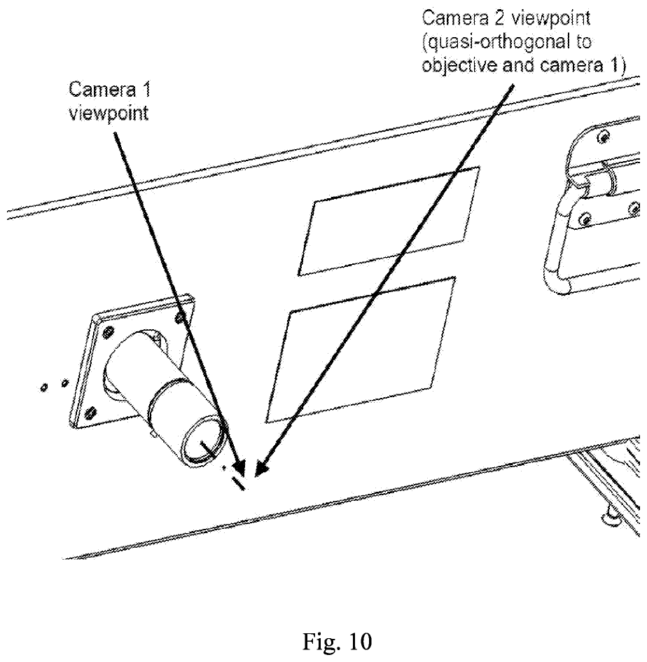

In an embodiment, coarse alignment of the mouse to the objective can be aided by one or more cameras (FIG. 10), each aligned to the working point of the objective. These cameras would be orthogonal or quasi-orthogonal with respect to each other and the objective. If the mouse's eye is centered in both cameras, it would also be centered to the objective at the working distance. These cameras might be sensitive to NIR, permitting course alignment of the mouse without visible excitation. This is beneficial as specific mouse/animal protocols might limit the exposure of the mouse's/animal's eyes to light for experimental purposes. More generally, eyes are more susceptible to damage from visible wavelengths than infrared wavelengths of similar incident power.

Prior to placing the mouse, it is helpful to center the positioner 701 relative to the objective 224, especially for the first use after the installation of the system.

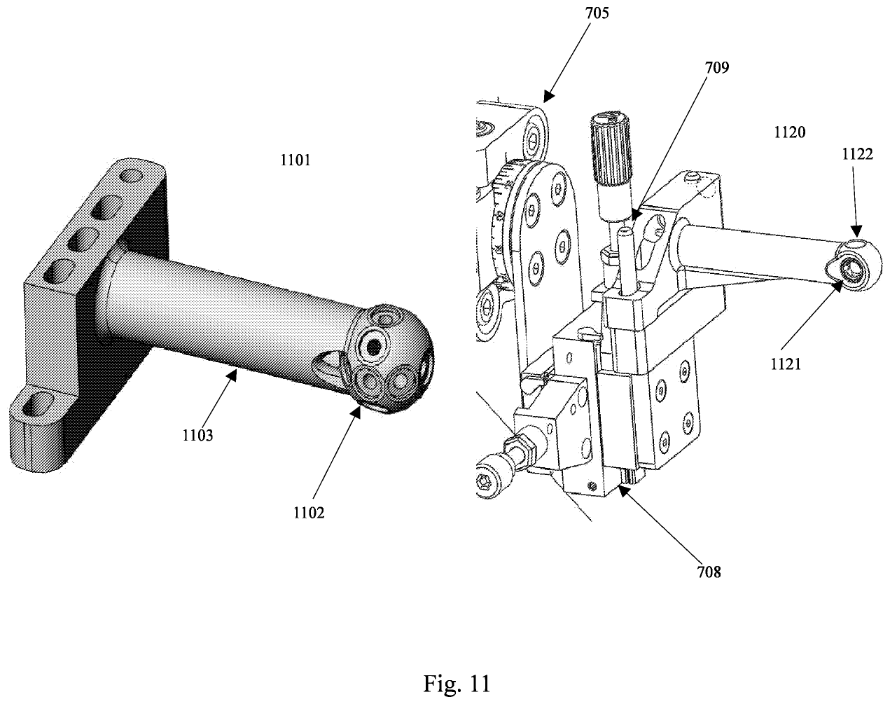

To assist with the centering of the positioner 701, in an embodiment, an alignment ball tool 1101 can be provided (FIG. 11). This alignment tool can have multiple orthogonal holes 1102 and a base that connects with the mounting pins 709. The holes are positioned such that their common axes will match the position of the mouse's eye. Without the tool, X and X′ are confounded, as are Y and Y′, etc. To use the tool, the system can be set to project a centered eye-safe alignment beam of visible light. Then X and X′ can be adjusted such that the beam is centered in the hole.

In an embodiment, an XY (i.e., Cross like) scan pattern is helpful, as it traces crosshairs that can be easily centered in the hole. At this point, the errors in X and X′ will cancel each other out, and so will be equal. For example, if the X stage 705 were off center from the objective 224 by 3 mm to the right, and the target ball 1101 was centered, then the X′ stage 707 would need to be off center by 3 mm to the left. The positioner can then be rotated around the nasal/temporal (i.e. Y) axis by 180 degrees. This changes the direction in which the X′ error acts. The beam will then be displaced from the center in the X direction by the sum of the equal X and X′ errors. In the previous example, after rotation, the X′ stage 707 would now be off center by 3 mm to the right, and the target ball 1101 would be off center by 6 mm to the right. The X error will then be half of the beam misalignment, as will the X′ error. In an embodiment where the X stage 705 has a quantitative indicator such as a micrometer knob, while the X′ stage 707 does not, X and X′ can be centered with a four-step process. First, the X′ stage 707 can be adjusted to center the target ball 1101, and the position of X recorded. Second, the positioner 701 is rotated to reverse the direction in which the X′ stage 707 acts. Third, the X stage 705 is adjusted to re-center the target ball 1101. In the above example, the X stage 705 would have moved 6 mm to the right. Fourth, the new position of X is recorded. Next, the X stage 705 is set to the average of the two recorded positions (e.g. 3 mm to the left) and the X′ stage 707 is adjusted to recenter the target ball 1101.

Centering Y and Y′ can be accomplished with a similar procedure, except by rotating around superior/inferior (i.e. X) axis. As being off center in Y makes it difficult to check X centering, it is expedient to center both X and Y concurrently, rotating both axes at the same time.

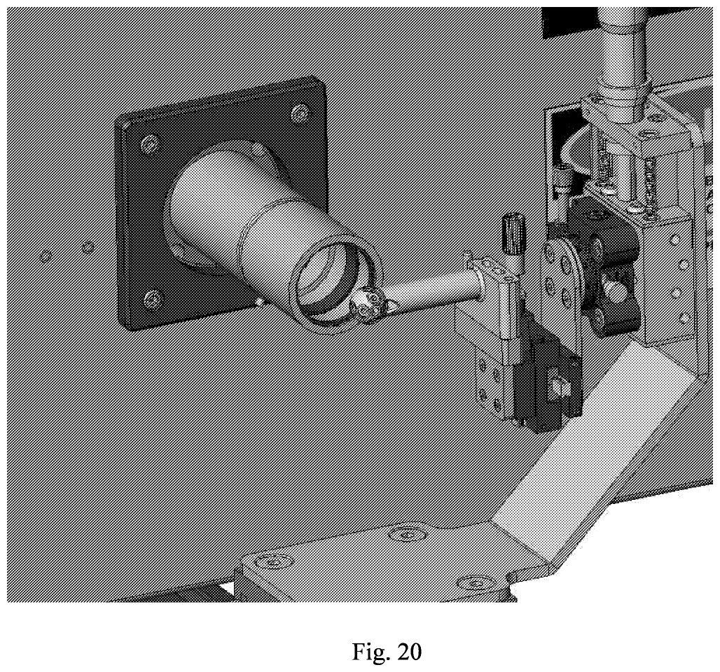

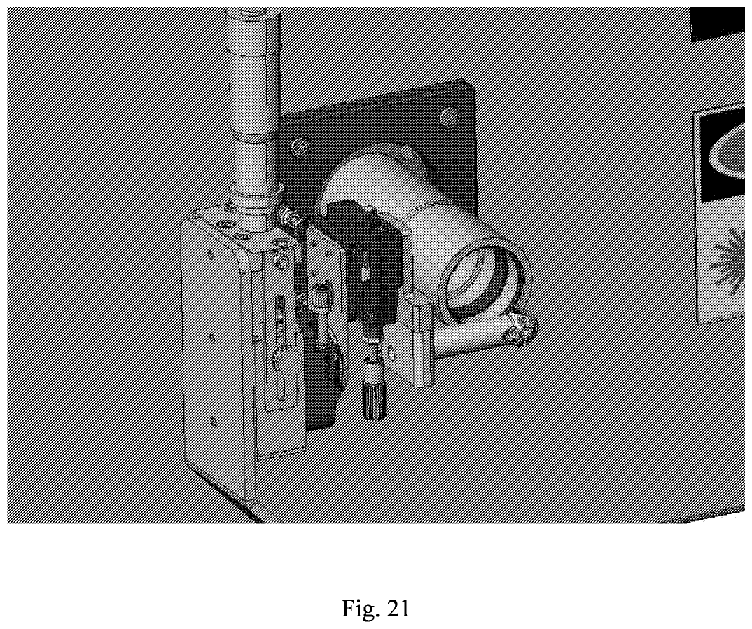

A procedure for centering X and Y using the target ball 1101 might be as follows:
a. Position the target ball on the positioner pins, as shown in FIG. 20. If this is the first time that the positioner has been centered, adjust X, Y, and Z to integer values close to the middle of their range.
b. Rotate both the nasal/temporal and superior/inferior axes to 0°, as shown in FIG. 20.
c. Turn on a low power, eye-safe, visible beam.
d. Start a scan to create horizontal and vertical lines, with two diagonal lines closing the loop. These will serve as crosshairs. The amplitude can be increased for a larger pattern, or decreased for a smaller one.
e. Focus the beam. Observe the beam on e.g. a sheet of paper on a near wall. Do not look directly into the objective.
f. If necessary, use the Z disengage to slide the positioner away from the objective, so that the corners of the pattern are blocked.
g. Use only X′ and Y′ to center the target ball on the cross. (It is expedient to start with X′ and Y′, since these won't involve moving X and Y out of position if previously aligned.)
h. Stop the scan, read the current values off the X and Y micrometers, and record them.
i. Rotate both the nasal/temporal and superior/inferior axes to 180°, as shown in FIG. 21. After rotations, the target ball might be off center. This is because X′ and Y′ were set to cancel out any error in X and Y in step 10. This would be because the rotations change the direction that the X′ and Y′ misadjustments acted in. Previously they canceled out the X and Y misadjustments. After the rotation, the misadjustments add to each other.)
j. Restart the scan. Using only X and Y, adjust the positioner to center the target ball on the cross.
k. Stop the scan, read the current values off the X and Y micrometers, and record them.
l. Calculate the average values for X and Y and record them. Adjust X and Y to these average values. This will set X and Y.
m. Press Start. (To turn the beam back on.)
n. Restart the scan. Using only X′ and Y′, center the target ball on the cross. This will set X′ and Y′.
o. Check the adjustment by rotating nasal/temporal and/or superior/inferior axes back to 0° degrees. The target ball should remain centered on the cross.

In an embodiment, this alignment ball tool 1101 can also be used to set Z′. Similar to X and X′, Z and Z′ are conflated. However, by rotating the superior/inferior (i.e. X) axis stage 706 by 90 degrees, as in FIG. 21. The vertical position of the target ball tool 1101 becomes the result of Y and Z′. If Y has been previously centered, then Z′ can be independently centered by adjusting Z′ on the Y′Z′ stage 708.

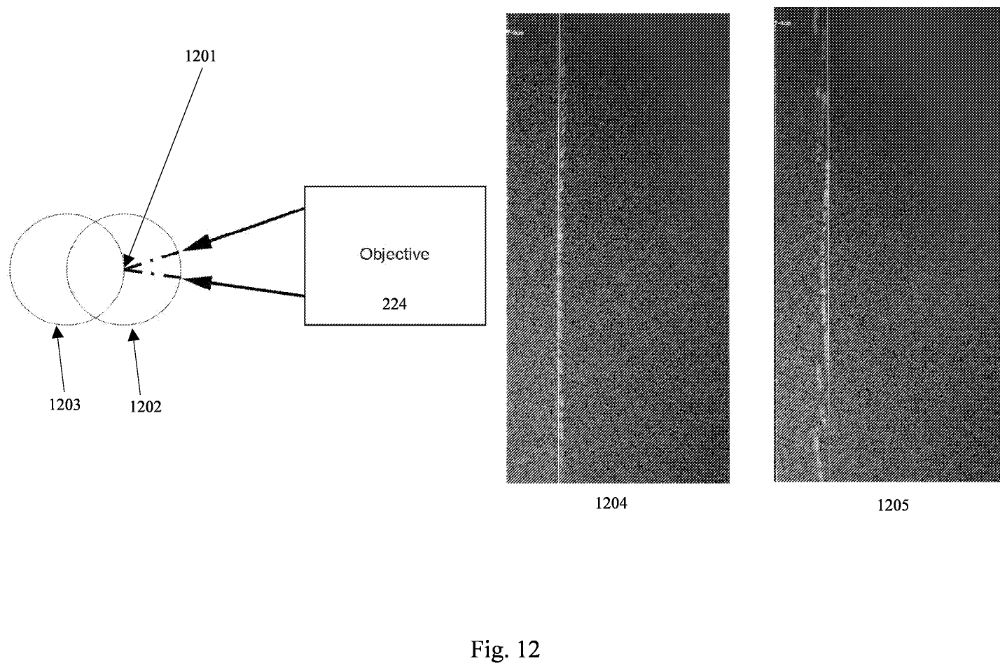

In another embodiment, the alignment ball tool 1101 can also be used to set Z if the objective 224 is such that the path traced by the scanner converges at some nodal point 1201 (FIG. 12). If the objective has such a point, the mouse will need to be positioned precisely relative to that point.

In such an embodiment, this precise positioning is necessary for the OCT cross sections in X and Y to be straight and vertical, and for the en-face views (from raster or other 2D OCT or SLO scans) to be in uniform focus across their surfaces. (The X and Y views display the X or Y axis on the vertical axis, and the optical path length, relative to the reference arm position, on the horizontal axis. Points with the same optical path length would be vertical to each other in the OCT B-scan images.) These straight and vertical X and Y cross sections might also be seen in the OCT image of a sphere centered at the nodal point of the objective. To set the positioner 701 to be at the objective's nodal point 1201, the underside of the shaft 1103 of the alignment ball tool can be imaged.

In an embodiment, a sphere 1202 is positioned with its center at the objective's nodal point. The cross section of the underside of the alignment ball tool shaft 1103 should then be vertical and flat, except for the steps from 3D printing (or any other manufacturing artifacts), as shown in the Y scan 1205. Z is set by setting XY and X′Y′Z′, and then adjusting Z until the Y-scan shows steps but is otherwise straight and vertical.

These steps, an artifact from 3D printing, are beneficial, as there is another position where the alignment ball tool 1101 shaft can appear to have a vertical B-scan. This position is analogous to a sphere positioned with its surface at the objective's nodal point 1203. The cross section of the alignment ball tool 1101 might then appear vertical and flat, as shown 1204. This position will deviate from the desired position.

In an embodiment, a lens target 1120 could also be provided. This has a small lens 1121 positioned to match the position and size of the mouse's eye. These lenses can emulate a mouse's or animal's eye, and so it could be used to practice aligning an animal eye. Additionally, it could be used to center the positioner.

In another embodiment, the target 1120 would use practices familiar to users experienced in aligning eyes to center the positioner 701. For example, instead of centering the excitation beam of an XY scan on a hole as done with the alignment ball tool 1101, the user could center the lens target as they would center a mouse eye. This is because a properly centered beam 1301, illustrated by a centerline and a node for simplicity, will result in a straight and vertical B-scan 1302. Were the centerline offset, e.g. in Y 1303, the resulting B-scan 1304 would be tilted but might be straight. Were there also an error in working distance—(Z or Z′), e.g. 1305, the resulting B-scan would be both tilted and curved, 1306.

To resolve Z from Z′, the lens target 1120 would need a right angle view 1122 either of the same lens or another lens set. This is because one procedure for Z assumes that Z′ has already been centered, and one procedure for centering Z′ involves rotating the superior/inferior (i.e. X) axis stage 706 by 90 degrees. This would present a right angle view of the lens target 1120. To center using lens target methods, this view would need to present a lens to the objective 224 when viewed from this angle.

In another embodiment, the positioner and main system can be integrated with sufficient accuracy and rigidity so as to be inherently centered. As centering might be effective if within e.g. 100 um, this might be attainable. (In contrast, subject alignment might need to be within e.g. 10 um for clear imaging.)

In another embodiment, the objective can be centered to the positioner.

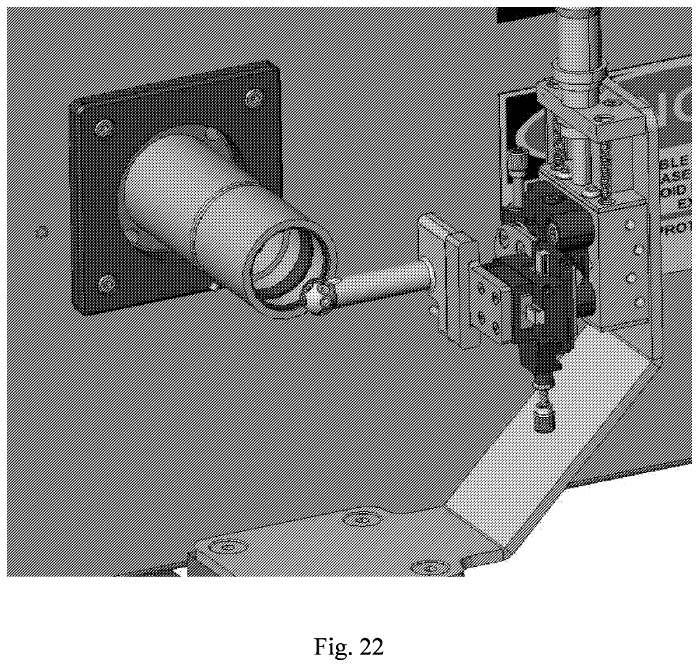

In another embodiment, the linear translation stages might have a mechanical indication of being in their middle position. For example, if X′, Y′, and Z′ could be set directly to their middle position, X, Y, and Z could then be roughly centered directly, e.g. X and Y by centering a ball target 1101, and Z by imaging the underside of the ball target shaft 1103 (see FIG. 22).

In another embodiment, stages could be set to their middle position by a separate tool. This tool might set one specific stage, many stages, or all stages at once.

In another embodiment, a positioner could have features necessary to automatically ensure the safety of the subject in a moving stage, and have motorized translation and/or rotation stages. Software could be used to center such a positioner automatically. For example, after manual or automated coarse alignment, the software could detect the subject's position by generalizing the retina in XY scans and then complete fine alignment of the subject. When instructed to rotate the subject around a specified axis by a specified amount, the software could watch for changes in the specimen's position and adjust the positioner centering to compensate. Such an implementation might have sufficiently precise control as to approximate a centered eight-degree of freedom positioner with a positioner that had only five degrees of freedom, and so could not be mechanically centered as discussed here.

In a further embodiment, centering features could be integrated into the cartridge. Such features might eliminate the need for a separate centering tool. The process of using such features might involve moving the positioner away from center to present the feature for imaging, as such a feature would need to not interfere with the subject. These features might be observed by the system's main optical functions (e.g. OCT or SLO) or with secondary alignment cameras (e.g. FIG. 10).

### Additional System Description

Angled animal cartridge: In an embodiment, the cartridge is angled so that the mouse eye axis is approximately aligned with the objective axis. See FIG. 8. Additionally, this angling aligns the nasal/temporal and superior/inferior axes with the system orthogonal axes, so the rotational axes are anatomically meaningful. That is, researchers wishing to rotate the mouse (or animal) to change the nasal/temporal or superior/inferior angle would be able to make the rotation directly, without needing to approximate the desired rotation using multiple rotational axes that are not relevant to the mouse's anatomy. See FIGS. 8 and 9.

The cartridges can be fabricated in different sizes to support the torso and head of the mice (or animals) of a variety of sizes. Side bolsters could be provided if necessary.

The cartridge can also provide for other means of securing the animal. These other means might include velcro straps, surgical tape, etc.

In an embodiment, this cartridge can be specific to the right or left eye, so that the eye is presented at a near optimal location irrespective of the size of the mouse or the eye to be viewed.

Alternatively, the cartridge can have two sets of mounting features, each corresponding to one eye, permitting ambidextrous use of the cartridge.

For mice and similar animals, this cartridge can be engineered for either the proptose or non-proptose eyeball position.

In an embodiment, the cartridge can provide an integrated heat source and temperature sensor that can rotate with the mouse. The heat source might be conductive with a resistance heater. It might alternatively be radiative with an IR emitter. (Specifically an IR emitter that does not interfere with near-IR system operation.)

In an embodiment, the heater can be controlled by a temperature sensor mounted at the heater. Alternatively, the heater can be controlled using feedback from a temperature sensor monitoring the animal's temperature, with a heater-mounted temperature sensor or switch used to avoid overheating in case of faults.

The mouse or animal temperature can be sensed from its skin, anus/rectum, ears or other body parts. The temperature sensor could be a non-contact infrared or visible light thermometer.

This cartridge can provide an integrated gas manifold to support gas anesthesia systems by providing orifices such as a gas inlet and outlet near the animal's nose. This inlet and outlet might be symmetric left-right. Alternatively, the inlet and outlet might be asymmetric, for example, having a dual outlet near the animal's jaw, and a single inlet near the nose, to provide flow over the animal's mouth as well. This can be useful in case the angle of the animal causes an exception to an animal's generally-nasal respiration. This gas anesthesia manifold and/or the orifices (inlets and outlets) would translate and rotate with the animal cartridge (and indirectly with the animal mounted on the cartridge). This is the key advantage of the proposed invention. See FIG. 8.

In an embodiment, this gas anesthesia manifold might be integrated into the cartridge or bite bar or a palate bar. It might use barb fittings.

This cartridge can secure the head via a bite bar or a palate bar, which will provide a bar that the mouse's or animal's incisors can be hooked on to, or a hole that the upper incisors can be inserted into. The animal's head might otherwise rest on the cartridge.

In an embodiment, this cartridge can secure the head via a palate bar. This bar will have a hole that the upper incisors can be inserted into, as well as steps that engage the mouse's or anima's molars or support the palate.

In another embodiment, the palate bar can have integrated features that can be used to depress the lower jaw, to facilitate placing the mouse's or animal's head securely on the palate bar. See FIG. 8.

In one more embodiment, this cartridge can secure the head via a chin rest.

In yet another embodiment, this cartridge can secure the mouse's or animal's head down against the bite bar, palate bar, or chin rest by use of a flexible muzzle strap.

This flexible strap might conform to the mouse's or the anima's muzzle on one side, and to the ridge on the other, to enclose a volume around the mouse's or the animal's nose, to contain a flow of anesthesia gasses. Alternatively, an optional, perforated strap can be used to permit the mouse or the animal to breath room air in case a gas anesthesia system is not used.

In another embodiment, this strap might be replaced with some other flexible form that conforms to enclose a gas flow. The strap can optionally be secured by thumbscrews or spring clips.

In a yet another embodiment, the strap can be replaced with a hook retainer which is secured by one spring clip. See FIG. 9. This hook retainer can be formed to partially enclose a volume around the nose to support the use of gaseous anesthesia. Alternatively, it can be fabricated with a slot to permit breathing room air freely, in the absence of gaseous anesthesia.

Alternatively, the mouse's or the animal's head might be secured upwards against an integrated muzzle arch.

These cartridges can be mounted so that they can easily be mounted or removed without tools. For example, the cartridge might have a hole and slot that slides down onto two pins on the holder arm. See FIG. 8.

These cartridges can be fabricated via 3D printing, such as via SLA in a waterproof resin, to facilitate customization. See FIG. 8.

The cartridges can also be manufactured by other means than 3D printing; e.g., injection molding or machining or many other processes.

XYZ and X′Y′Z′: In an embodiment, to permit a larger region of the mouse/animal eye to be imaged than can be viewed from any one perspective, this holder can optionally provide for the three-fold alignment of the objective's working point, the intersection of the rotational axes, and the corresponding focal node of the mouse's or animal's eye. It is useful to have 8 degrees of freedom. 3 degrees of freedom are helpful to exactly align the intersection of the holder's rotational axes to the objective. These can be provided by X, Y, and Z stages that are not mounted on the rotational stages. 3 degrees of freedom are useful to adjust the cartridge position to compensate for small variations between mice/animals. These can be provided by X, Y, and Z linear translation stages that are mounted on the rotational stages, which can be called X′, Y′, and Z′. See FIG. 7.

A further degree of freedom might be helpful to disengage the holder arm from the objective when changing cartridges, and to re-engage to a repeatable stop position. Two rotational degrees of freedom are helpful to align the animal eye to the beam nodal point.

To reduce mechanical complexity, stages parallel to axes of rotation can be mounted either on or off that rotational frame. Otherwise, three right-angle brackets might be needed; one each for XYZ, the two rotations, and X′Y′Z′.

In another embodiment, to assist with the centering of the stage, an alignment ball tool can be provided. This alignment tool can have multiple orthogonal holes. Without the tool, X and X′ are confounded, as are Y and Y′, etc. To use the tool, the system can be set to project a centered eye-safe alignment beam of visible light. Then X and X′ can be adjusted such that the beam is centered in the hole. At this point, the errors in X and X′ will cancel each other out, and so will be equal. The tool can then be rotated around the nasal/temporal axis by 180 degrees. This changes the direction in which the X′ error acts. The beam will then be displaced from the center in the X direction by the sum of the equal X and X′ errors. The X error will then be half of the beam misalignment. See FIG. 11.

In one embodiment, this process can be automated. For example, the software can estimate the positional error in one orientation, then instruct the operator to rotate a specific axis 180 degrees (or the axes could be rotated by the software as well), and then estimate the new error. The correct X or Y adjustment will eliminate the mean error.

This alignment ball tool can also be used to set Z if the objective is such that the path traced by the scanner converges at some nodal point. If the objective has such a point, the mouse will need to be positioned precisely relative to that point. This precise positioning is useful for the OCT cross sections in X and Y to be straight and vertical, and for the en-face views to be in uniform focus across their surfaces. These straight and vertical X and Y cross sections might also be seen in the OCT of a sphere centered at the nodal point of the objective (i.e. radial alignment). However, if the sphere had a surface with a diffuse reflection, these straight and vertical X and Y cross sections might also be seen if the sphere's surface was at the objective's nodal point (i.e. point alignment). To avoid this ambiguity, the surface could be produced to have a specular reflection, it could be distinctly textured, or a cylindrical surface could be used. A sphere with a specular reflection may not reflect light back to the objective if the sphere's surface was at the objective's nodal point. However, placing objects with specular reflections in high energy light paths might be contrary to best practices.

Alternatively, a distinctive surface texture, such as the steps inherent in 3D printed parts, could be used to confirm that the user is seeing a radial alignment instead of a point alignment. In the case of a radial alignment, the distinct steps would be visible in the cross section, as a radial section was getting imaged. In contrast, in the case of point alignment, the steps would not be seen, as only one point was being imaged.

In an alternate embodiment, a cylindrical surface could be used. Radial alignment might be achieved only in the cross section that was sectioning radius. The other cross section, viewing the straight length of the shaft, would not be expected to show radial alignment (or not show it equally). In contrast, both cross sections would show point alignment equally. The alignment ball tool shown in FIG. 11 includes both 3D printed steps and a cylindrical surface along its shaft. (The X′ axis of the mouse positioner can be used to bring the cylindrical shaft into view of the objective.)

This alignment ball tool can also be used to set Z′. By rotating superior/inferior by 90 degrees, the Y/Y′ position of the target ball becomes the result of Y and Z′. If Y has been previously centered, a similar procedure can be used to center Z′ in turn.

Lens target: A practice target can be provided with one or more lenses. In optical imaging, these lenses can be superficially similar to the mouse's or animal's eye. They can be used to become skilled at operating the mouse or animal positioner without requiring a mouse or an animal. See FIG. 11.

Such a lens target might also be utilized to center the positioner. This can serve as an alternative for the operators so they may not need to learn the steps to center the ball tool, as well as steps to center mouse eyes and lens targets.

The mouse positioner can be modified to mount rats, squirrels, other small animals, zebra-fish, tadpoles, mid-sized and large animals.

Subframe: For clear imaging, the mouse or animal positioner can be rigidly mounted with respect to the main system. This can be accomplished by screwing the positioner down to a large (optionally optical) table with tapped holes, and placing the system on that table. Alternatively, an optional subframe can be mounted to the system. This subframe can hold the positioner rigidly relative to the objective. See FIG. 7.

Stand: A stand can be provided to hold the mouse or animal cartridge upright for mouse or animal insertion and preparation. These steps are easier when the mouse or the animal is at a horizontal level. Once complete, the cartridge and mouse/animal can be transferred from the cartridge stand onto the positioner.

### Method of Image Acquisition and Analysis

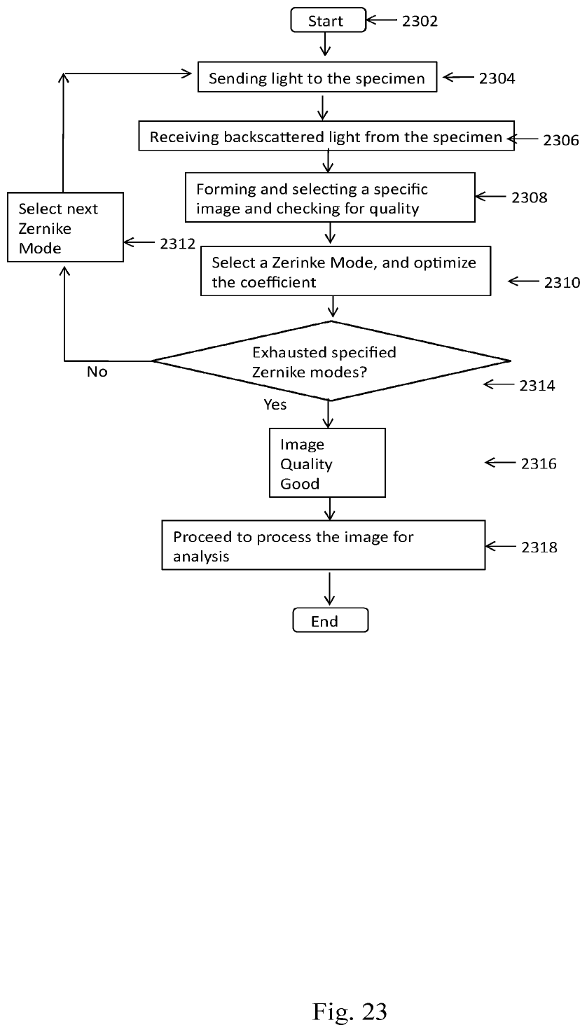

FIG. 23 shows a method to perform sensorless adaptive optics. Images are acquired from the specimen using OCT or OCT-angiography or SLO or Doppler OCT (or all of these technologies in some embodiments). A variety of figures of merit (FoM) can be computed to determine the image quality. The amplitude of each Zernilke mode for the deformable mirror is optimized to get the best FoM and the best amplitude for each Zernike mode for the mirror. Those Zernike mode amplitudes are set for the deformable to get an optimal mirror shape. Thus optimal image quality is obtained for the sensorless adaptive optics imaging mode.

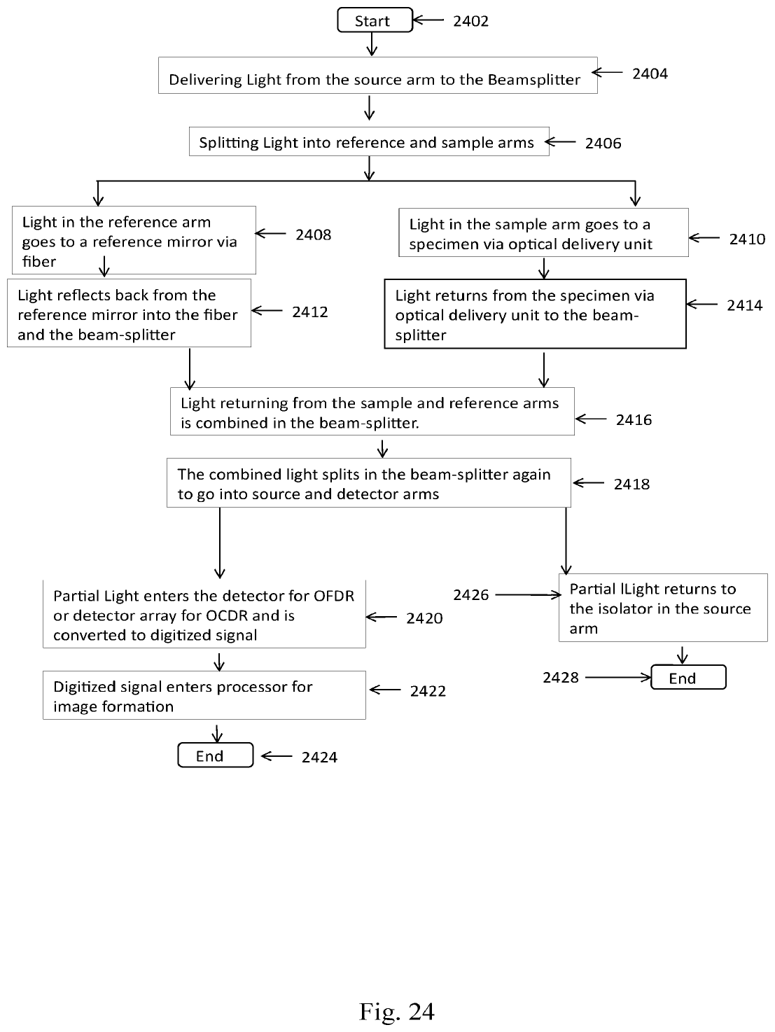

FIG. 24 describes a method of acquiring an image from a specimen using the OCDR-OCT sub-system. A light source may be a tunable light source, a broadband source, or a laser. An apparatus or system is used to send a specific bandwidth light from a light source to a specimen using a source arm and sample arm. A backscattered light from the specimen is received by the optical delivery unit. An image is formed after going through the grating and detector array and checked for quality. If the image quality is poor, the steps from are repeated. If the image quality is good data is further sent to produce an image for analysis using the processor algorithms. The process ends once the image is formed. The system comprises of the optical delivery unit 108 in the sample arm.

FIG. 24 describes the steps of light travelling through the source to the specimen and the signal from the light being processed. Light is being delivered using a light source using the sample arm to the beam splitter 2404. Beam splitter splits the light into two parts sending the first path light to reference arm 2408 and second path light into the sample arm 2410. The second path light goes to the specimen via the optical delivery unit. The specimen in this case may be a human or an animal eye. Blood flow creates fluctuating intensity backscattered light. Regular stationary tissue creates varying but non-fluctuating light. Light from the stationary tissue as well as blood flowing areas are returned to the beam splitter.

Sample arm sends the second path of light to the specimen (or the eye) using the optical delivery unit and the specimen (or the eye) reflects back the second path of light as a returning light via the optical delivery unit to the beam splitter 2414. A reference mirror returns the light into the fiber to be combined with the returning light from the specimen at the beam splitter 2416. Thus, the reference mirror in the reference arm returns the first path light to the beam splitter to join a returning light from the eye or the specimen. The combined light splits in the beam splitter again to go into source and detector arms 2418. A partial returning light from the beam splitter travels through a detector arm to a grating unit and a detector array in OCDR-OCT system or enters the detector if it is OFDR-OCT system to be converted to digitized signal 2420 using analog-to-digital-converter 624. Digitized signal enters the processor for A-scan generation and/or image (B-scans) formation 2422. The method ends there 2424. On the other hand partial light returns to the isolator using the source arm 2426 and the method ends there 2428. The system comprises of the optical delivery unit 408 in the sample arm.

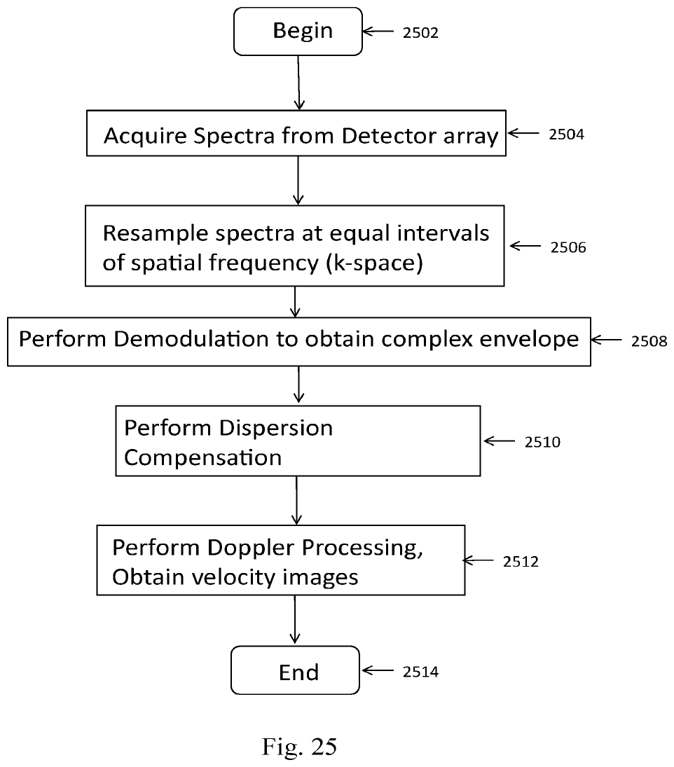

FIG. 25 shows a high level flow of the processing algorithms. Step 2502 is the beginning step. For the OCDR-OCT system, the spectra are acquired from the detector array as explained earlier (Step 2504). Since the acquired spectra are typically spaced in equal intervals of wavelength, in the step 2506, the spectra are resampled at equal intervals of spatial frequency (k-space) using a frequency resampling algorithm. Next in step 2508, demodulation, which includes inverse Fourier transforming, is performed to extract the complex envelope of the signal. Next in order to correct for the dispersion in the system, the dispersion compensation is performed in step 2510. Next in step 2512, Doppler processing is performed to extract velocity images. The method ends in step 2514. These algorithms are processed in a processor 254 and displayed as a gray scale or pseudo-color image. By way of example, not by limitation, this processor can be a computer, Field Programmable Gate Array (FPGA), an embedded system or a microcontroller.

Frequency Resampling: The spectra W_{ccd}(λ,x) measured by the spectrometer (i.e., the output of the digital array) are equally spaced in wavelength (λ). However in order to obtain an accurate A-scan measurement by inverse Fourier transforming, the spectra need to be re-measured at equal intervals of spatial frequency (k=1/λ). Thus, if N is the total number of samples, the spectra are measured at equal intervals in wavelength δλ=(λmax−λmin)/N. The spectra need to be equally spaced in k-space. Thus, if the corresponding maximum and minimum wavenumbers are kmax=1/λmin and kmin=1/kmax, then the spectra need to be re-sampled at equal intervals in k given by δk=(kmax−kmin)/N to obtain S_{ccd}(k,x). If the data are over-sampled while re-sampling by a factor of X, then δk=(kmax−kmin)/XN.

There are many algorithms for re-sampling the spectra. One such method is simple linear interpolation as described by [Vergnole et al 2010]. Thus, if we need to calculate the spectrum S_{ccd}(k₀,x)at a location k₀, and the spectra are measured at the nearest neighboring wavenumbers kᵤ (upper wavenumber=1/λᵤ, λᵤ is the upper wavelength), kₗ (lower wavenumber=1/λₗ, λₗ is the lower wavelength), then

$${S}_{ccd} ( {k}_{0} ) = {S}_{ccd} ( {k}_{l} ) + {U}_{0} [ {S}_{ccd} ( {k}_{u} ) - {S}_{ccd} ( {k}_{l} ) ] ; {U}_{0} = \frac{{k}_{0} - {k}_{l}}{{k}_{u} - {k}_{l}}$$

and note that S_{ccd}(kₗ)=W_{ccd}(λₗ, x)=and S_{ccd}(kᵤ)=W_{ccd}(λᵤ, x)
Another method described by [Vergnole et al. 2010] is spline interpolation. A preferred and faster method of interpolation is achieved by convolution using a Kaiser-Bessel window as described by [Vergnole et al. 2010].
S_{ccd}(k₀)=Σ_{l=-M/2}^{M/2} S_{ccd}(kₗ)C₀(kₗ) where kₗ are the non-linearly placed neighboring values of wavenumbers, M is the size of the convolution kernel. M can be any value, however a value between 3 to 9 can yield good results.

$${C}_{0} ( {k}_{l} ) = \frac{{I}_{0} ( γ \sqrt{1 - {( \frac{2 H}{M} )}^{2}} )}{M}$$

where H=smaller of

$$\frac{M}{2}$$

or (k−kₗ)/δk and I₀ is the zero-order Bessel function of the first kind.

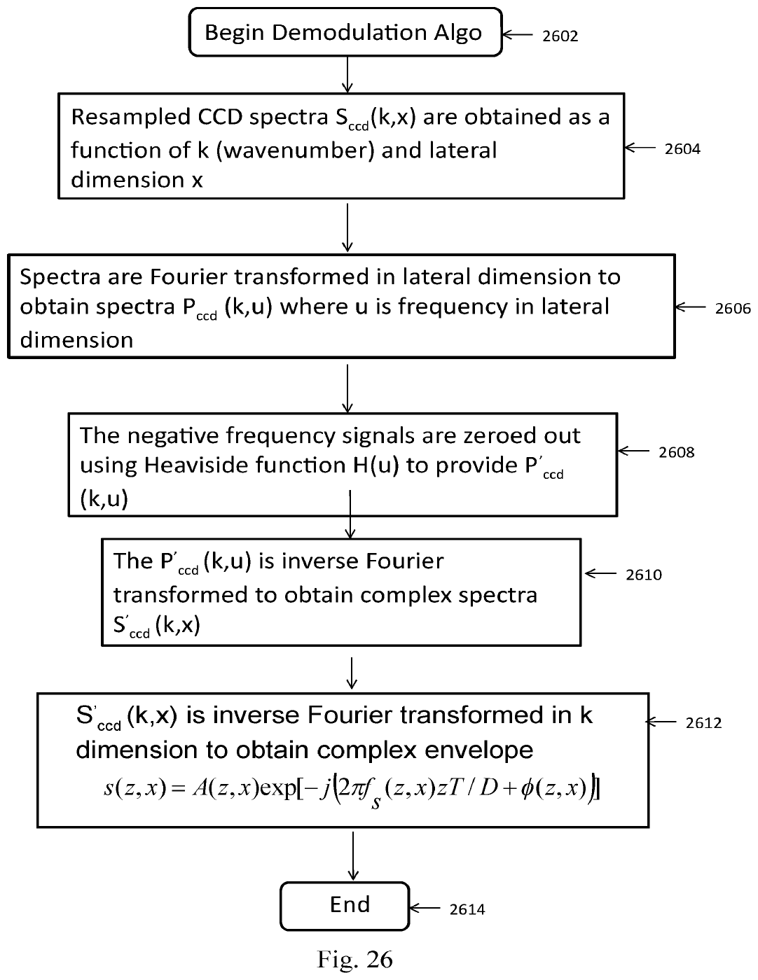

Next in FIG. 26, we present a novel algorithm such as a demodulation algorithm (step 2602), which is also instant version of the modified Hilbert transform algorithm:
1) Resampled CCD spectra S_{ccd}(k,x) are obtained as a function of k (wavenumber) and lateral dimension x (step 2604).
2) Spectra are Fourier transformed in lateral dimension to obtain spectra P_{ccd}(k,u) where u is frequency in lateral dimension (step 2606).
3) The negative frequency signals are zeroed out using Heaviside function H(u) to provide P′_{ccd}(k,u) (step 2608).
4) The P′_{ccd}(k,u) is inverse Fourier transformed to obtain complex spectra S′_{ccd}(k,x) (step 2610).
5) S′_{ccd}(k,x) is inverse Fourier transformed in k (i.e., depth) dimension to obtain complex envelop in Eq. 2 (step 2612)

s(z,x)=A(z,x)exp[−j(2πfₛ(z,x)zT/D+ϕ(z, x))]. (Eq 6)

Here A(z,x) is the amplitude of the detected signal corresponding to the depth-resolved reflectivity obtained in conventional OCT imaging and ϕ(z, x) is the phase corresponding coherent interference of backscattered waves, commonly known as speckle. Here z is the depth location, x is the lateral location, D is total depth of A-scan, T is the time taken to acquire an A-scan. For a broadband source, A(z,x) is a highly localized function (e.g., a Gaussian) whose width determines the axial resolution of the OCT image. fₛ is Doppler shift in light backscattered from moving objects in the sample. A scatterer in the sample moving with a velocity Vₛ induces a Doppler shift in the sample arm light by the frequency

fₛ=2Vₛ[cos θ]nₜv₀/c (Eq. 7)

where θ is the angle between the sample probe beam and the direction of motion of the scatterer, nₜ is the local tissue refractive index, v₀ is the source center frequency, and c is the light velocity.

Dispersion compensation: Group velocity dispersion needs to be matched between the reference and sample arms. In some embodiments of the instant invention, dispersion is compensated numerically by flattening the Fourier domain phase of a mirror reflection. Current proposed procedure comprises of:
a) Measuring the interferogram by placing a mirror in the sample, computing the complex envelope mₛ(z)=Aₘ(z)Exp(jφₘ(z)) (Here z is distance in depth, Aₘ is amplitude and φₘ is phase) for the interferogram. Such an intrerferogram can also be measured by removing the sample/specimen in the sample arm.
b) Computing the complex envelope for each interferogram measurement for any desired specimen as described in FIG. 26.
c) Multiplying the complex envelope by Exp(−jφₘ(z)) to perform dispersion compensation.

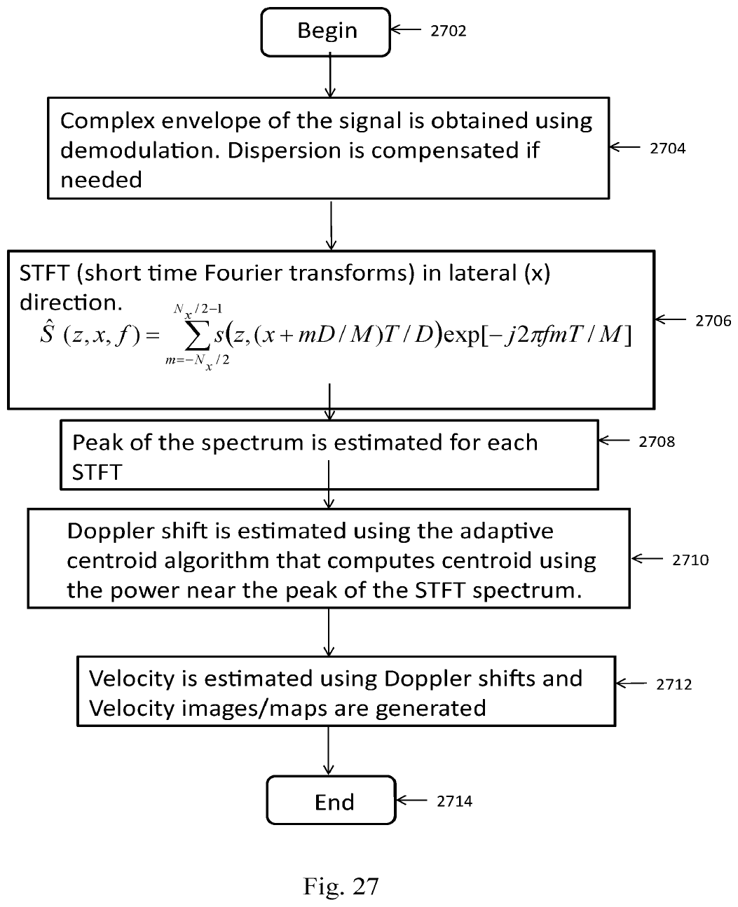

Coherent Deconvolution or complex deconvolution for Dispersion Compensation: Another process known as coherent deconvolution. The coherent deconvolution process comprises of
a) Measuring the interferogram by placing a mirror in the sample, computing the complex envelope mₛ(z)=Aₘ(z)Exp(jφₘ(z)) (Here z is distance in depth, Aₘ is amplitude and φₘ is phase) for the interferogram. Such an intrerferogram can also be measured by removing the sample/specimen in the sample arm.
b) Computing the Fourier transform of mₛ(z) to obtain Mₛ(k), where k is spatial frequency,
c) Computing the complex envelope s(z,x) for each interferogram measurement for any desired specimen,
d) Computing the Fourier transform of s(z,x) to obtain S(k,x),
e) Dividing S(k,x) by Mₛ(k) to obtain S₁(k,x),
f) Multiplying S₁(k,x) by a Wiener filter to obtain S₂(k,x) and
g) Computing inverse Fourier transform to obtain dispersion corrected sample measurement s₂2(z, x).
   h) In FIG. 27, Doppler processing algorithm for high accuracy and high precision velocity estimation is described (step 2702). The data set resulting from the camera can be processed in the processor 414 by the proposed Doppler algorithm which computes STFT (short time Fourier transforms) in lateral (x) direction (step 2706).

$$\hat{S} ( z , x , f ) = {\sum }_{m = - {N}_{x} / 2}^{{N}_{x} / 2 - 1} s ( z , ( x + m ) T ) exp [ - j 2 π fmT ] \qquad ( Eq 8 )$$

where Nₓ is the number of A-scans in the STFT window. Next the peak of the STFT spectrum is estimated (step 2708). Next, the Doppler shift is computed by an adaptive centroid algorithm (which computes centroid using the power near the peak of the STFT spectrum) (step 2710). Next, the velocity is estimated using Doppler shifts and Velocity images/maps are generated (step 2712). Step 2714 is the end of Doppler processing. The velocity precision is given by

Vₛ^{up}=c/(2NₓTv₀nₜ cos θ) (Eq 9)
i) Doppler shift algorithm is used for estimating Doppler shifts by computing the centroid of the short time Fourier transform spectrum using power near the spectral peak, which is an adaptive centroid algorithm. As we can see, velocity precision is higher with higher T (A-scan acquisition period). Therefore, in order to detect micro-flow (˜100 to 800 microns/s speed) in capillaries, by way of example but not by limitation, we can choose an A-scan rate of e.g., 2560 A scans/s. The maximum retinal blood flow velocities typically range to 1-4 cm/s. By way of example but not by limitation, higher velocities can be measured by performing another scan at a much higher speed of 42000 A scans/s. By way of example but not by limitation, from Eq. 4, choosing Nₓ between 1 to 30, we can measure velocities as low as 15 mm/s to 0.5 mm/s, respectively. By way of example but not by limitation, we can scan retina at 2 different scan rates, viz., 2560 A scans/s and 42000 A scans/s. By way of example but not by limitation, in the first set, we can scan 10 concentric circles centered at the optic disc, each comprising of 100 A-scans, which can be acquired in 4 seconds. By way of example but not by limitation, the second set would be acquired at the same locations, 10 concentric circles, each consisting of 420 A-scans, which can be acquired in 1 s. The scanning may be performed by the disc of the retina by performing concentric circles at a variety of speed. Optical delivery unit in the sample arm creates scan patterns, wherein the scan-pattern comprises of at least two B-scans, each B-scan having its specific A-scan rate.
j) Thus, we propose scan-patterns comprising of at least two B-scans wherein the first B-scan's A-scan rate is slower than the A-scan rate in the second B-scan.
k) In an embodiment, the scan-pattern can comprise of at least two B-scans, each B-scan having its specific A-scan rate.
l) This Doppler processing step can used to estimate blood flow velocities for augmenting diagnosis of diabetic retinopathy. By acquiring B-scans at various locations, this can be used to obtain a 3-dimensional map of blood flow velocities or blood vessels in the retina as well as any organ of a human or animal body.
m) The method of FIG. 25 is also applicable for an OFDR-OCT system. In the OFDR-OCT system, the light entering the detector arm from the beam splitter is incident on the detector and converts to an interferometric electric current or signal. The tunable light source produces a light of various frequencies within a specific bandwidth. This frequency/wavelength sweeping is performed at a very high speed and the detector is able to measure the interference signal at each of the frequencies. Such a high speed measurement produces a spectrum for further processing (step 2504 in FIG. 25). These spectra are typically measured at equal intervals of wavelength. Therefore, the spectra measured by the detector are processed using a re-sampling algorithm. Thus, the spectra are resampled at equal intervals of spatial frequency (k-space) (step 2506). There are some specialized OFDR-OCT systems where the source is able to sweep the bandwidth at equal intervals of spatial frequency (k-space). In those cases, the resampling algorithm is not needed. Next the signal is demodulated to extract its complex envelope (step 2508) and generate A-scans. The absolute part of the complex envelope (A-scan) is traditional OFDR-OCT signal. Next, the dispersion compensation is performed so that the signal has better depth resolution and higher fidelity (step 2510). Finally, Doppler processing is performed to obtain velocity images, which has velocity information within various locations within a specimen (or an eye) (step 2512).

### Additional System Description

OCDR-OCT system and OFDR-OCT are able to image sub-surface retinal microstructure and has been useful for diagnosis and management of diabetic retinopathy.

In some other embodiments, the system is used for optical coherence tomography (OCT) imaging and the OCT system comprises of a depth-scanning reference mirror to implement time-domain OCT (as described in Huang et al 1991, Fercher 1996, U.S. Pat. No. 5,321,501).

In some embodiments, the resolution of the OCT system improved using adaptive optics.

In other embodiments, the resolution of the fluorescence system is improved using adaptive optics.

In some embodiments, flow measurements (e.g., OCT Doppler, or OCT-angiography) are performed in addition to the fluorescence measurements.

In some other embodiments, fluorescence Doppler measurements are performed to measure the flow.

In some embodiments, contrast agents or fluorophores are introduced in the specimen to generate fluorescence.

Various materials can be used to construct the apparatus/system. Some examples are (not by limitation) 6061 aluminum alloy (i.e., UNS A96061) and its various varieties including (but not limited to) 6061-O, 6061-T4, 6061-T4 etc.; Nickel-chromium alloys such as INCONEL® (a registered trademark of the INCO family of companies) alloy 600; stainless steel and related alloys (e.g., UNS N02200, UNS N02201, UNS N04400, UNS N06600, UNS N06625, UNS N08800, UNS N08825, UNS N10276, UNS N08020, etc.) heat and chemical resistant polymers such as TOPAS® COC (by Topas Advanced Polymers). Acetal homopolymer such as Dupont's Delrin® can also be used as these polymers are tough, can sustain high stress and strain and are strong, and yet easily moldable.

Some more materials that can be used to construct the apparatus or system include (not by limitation) High-density polyethylene (HDPE), Polyvinyl chloride (PVC), Acrylonitrile butadiene styrene (ABS), Polyether ether ketone (PEEK).

The apparatus or system can be built (by way of example and not by limitation) by the process of reaction injection molding, which can produce high-strength, lightweight and flexible parts using thermosetting polymers such as polyurethane.

The apparatus or system could also be built (by way of example and not by limitation) by structural reaction injection molding (SRIM), where fiber meshes are used as a reinforcing agent.

The apparatus or system could also be built (by way of example and not by limitation) by injection molding, using thermoplastics or thermosetting plastics.

The apparatus or system could also be built (by way of example and not by limitation) by normal machining and assembly.

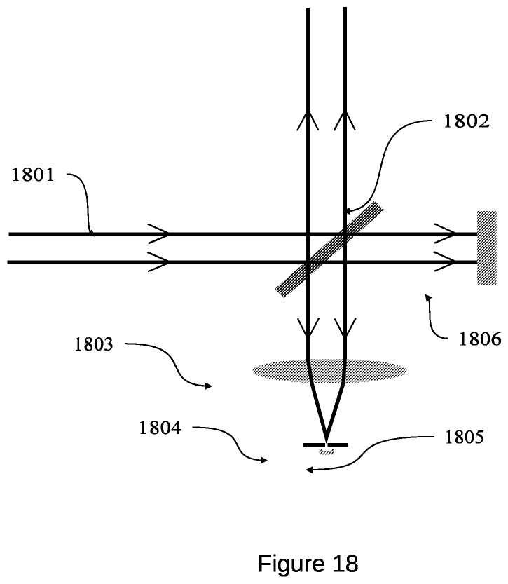

In another embodiment, the suppression of parasitic back reflections in a combined OCT/SLO system (or optionally, stand-alone SLO system) is performed by confocal reflectance detection with non-polarizing beam splitter (FIG. 18).

The animal retina is a structure with inherently low optical reflectivity. Imaging in the reflectance modality therefore requires very high signal to noise and therefore low parasitic back-reflection from imaging optics. A system combining OCT and SLO reflectance imaging introduces constraints on techniques relying on polarization selective optical components. A system operating over a wide optical bandwidth introduces constraints on the performance of any anti-reflection coatings.

The Illumination and detection of SLO comprises of an optical system comprising Collimated light source 1801. Non-polarizing plate beam splitter 1802 with high transmission of incident illumination, an imaging objective 1803, a pinhole, located at the focus 1804, a high gain optical detector such as an avalanche photodiode or a photo-multiplier tube (PMT) 1805 located closely behind the pinhole.

The system further comprises of a beam dump 1806 for absorption of a large fraction of the transmitted beam, with minimal scattering.

Imaging objective 224 and other optical elements in the system make use of double concave/convex imaging elements and assemblies with no flat sides, minimizing the fraction of reflection while maintaining the same collimation as the incident illumination.

In another embodiment, the excitation and fluorescence light paths are directed through a fluorescence filter cube (FFC). The FFC comprises of an excitation filter, emission filter and a dichroic beam-splitter. The FFC can enable multiple excitation and emission bands. Multiple FFCs can be put on a translator (such as a stepper motor) to provide more excitation/emission bands.

The fluorescence detection and OCT/OCDR detection can be combined using a dichroic mirror. One example of a dichroic would permit reflection of fluorescence light and transmission of the OCDR light. Another example would be transmission of fluorescence and reflection of OCDR light. Another example would be permit only OCT or fluorescence detection at any one moment. These modalities could alternate very quickly (e.g., within a few milliseconds) or the operator could choose to switch at their preferred time.

Another example would be the use of OCT light to excite the fluorophores. The resulting fluorescence (which would have higher wavelength than the emission) would return from the sample and a dichroic which reflects a longer wavelength and transmits a shorter wavelength can be used to separate OCT and fluorescence measurements.

In an embodiment, the above options are provided in the same system, and the beam-splitters and/or mirrors are positioned on a translator to provide various modalities.

In an embodiment, the specimens can be animals such as mice and rats.

In an embodiment, an extra variable focal length lens is used in the OCT beam (before combining with the fluorescence beam) so that the focus of the fluorescence excitation/emission light matches with the incident OCT light. This is a method to minimize chromatic aberrations.

In another embodiment, an extra variable focal length lens is used in the fluorescence path (before combining the OCT beam) so that the focus of the fluorescence excitation/emission light matches with the incident OCT light. This is a method to minimize chromatic aberrations.

This would permit variable excitation wavelength and emission spectra measurements and still obtain the best images for OCT as well as fluorescence.

A fixed focal length lens could be used instead of the extra variable focal lens if the fluorescence beam spectra and OCT beam spectra are known exactly.

### Test Targets

Determination of coincident focusing and characterizing achievable optical resolution is required in a system which combines simultaneous fluorescence and OCT imaging modalities. Regular characterization is especially important where said system provides fluorescence excitation and detection across a wide spectrum of optical wavelengths. A common usage is in preparation for animal studies which seek to image and localize fluorescently tagged structures within an animal eye.

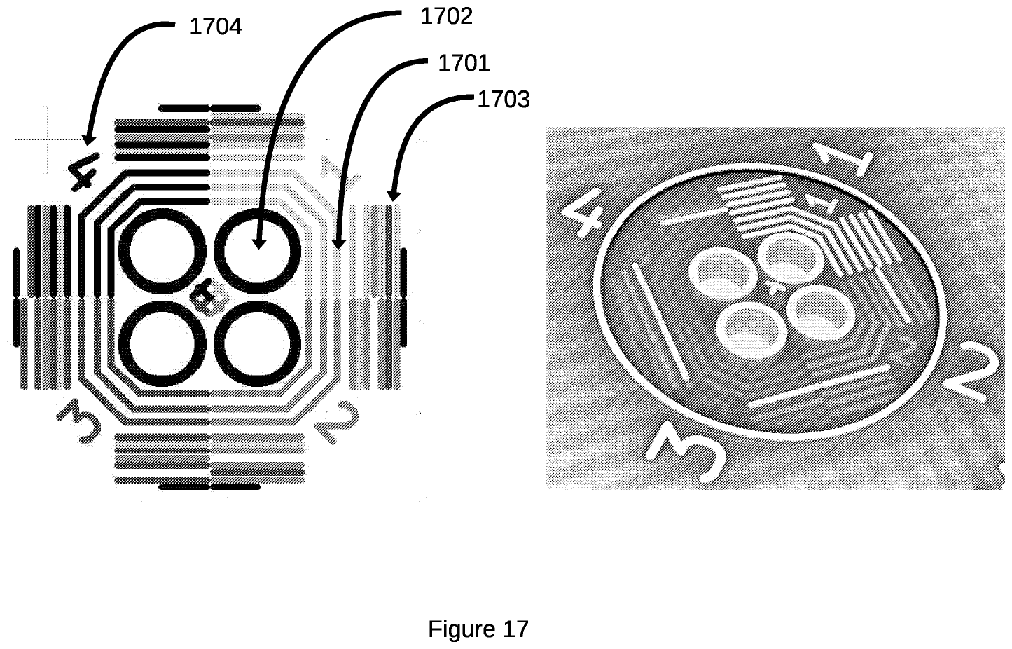

In an embodiment of the invention, a multi-layer test target (FIG. 17) is used to characterize imaging performance across multiple imaging modalities. The target is constructed with multiple layers of patterns formed in an opaque foil, each formed at different depths within a transparent or semi-transparent substrate. These patterns are referred to as metalization patterns.

In one embodiment, a multi-layer test target is fabricated with off-the-shelf printed-circuit-board (PCB) manufacturing technology, then having copper metalization layers and a substrate of glass-reinforced epoxy laminate, together forming a flat target.

In one embodiment, a multi-layer test target in the form of a flat disc or similar shape is assembled into a larger target assembly, wherein the focusing of an eye is emulated with an aspherical or spherical lens, the flat target being placed at or near to the focus.

Metalization patterns can consist of sets of parallel lines 1701, in one or more orientations.

Resolution at each depth can be determined by selecting each layer within an OCT cross-sectional-image (B-scan), generating an en-face-raster image, then extracting an intensity profile across the alternating metal lines. The impulse response function can be measured from this dataset by analyzing the intensity profile and can be used to determine the resolution.

In another embodiment, SLO imaging resolution could also be characterized using the metalization patterns on a multi-layer

Each metalization layer can be associated with a hole 1702 (which can be formed by drilling, or another method).

Each hole can be filled with a polymer resin (or a material that can serve as a transparent or semi-transparent host for holding fluorescent particles) up to the height of the associated metalization layer.

The hole can be further filled with a thin layer of fluorescent particles suspended in polymer resin.

The hole can be further filled to the top with polymer resin (or another host material).

In operation, a layer can be identified and focused in OCT, followed by focusing the coincident fluorescence modality to ensure coincident focus between the modalities.

Layers may be labeled numerically for identification 1704.

One or more sections of interspersed metalization lines may have the gap between lines filled with a metalization line on another depth layer 1703. This allows the degradation in resolution due to structures at depths outside the selected depth range to be characterized.

Interspersed metalization lines may permit the characterization of depth-insensitive imaging performance. With OCT depth selection disabled, a solid opaque region can be observed in regions where interspersed layers are present.

In another embodiment, a flat resolution test target is formed using a commercially available USAF-1951 resolution target, or similar standard resolution target. This standard resolution testing device consists of a glass slide with a series of black lines arranged in numbered groups, each providing a sample of various spatial frequencies for resolution assessment.

In one embodiment, the flat resolution test target is located at the focus of an aspherical or spherical lens, which emulates the focusing of an eye. This forms a test target assembly.

In one embodiment, the test target assembly can be screwed onto an Objective crown, which can in turn be installed on the front of the objective, rotated to any desired angle and secured with a set screw or similar fastener.

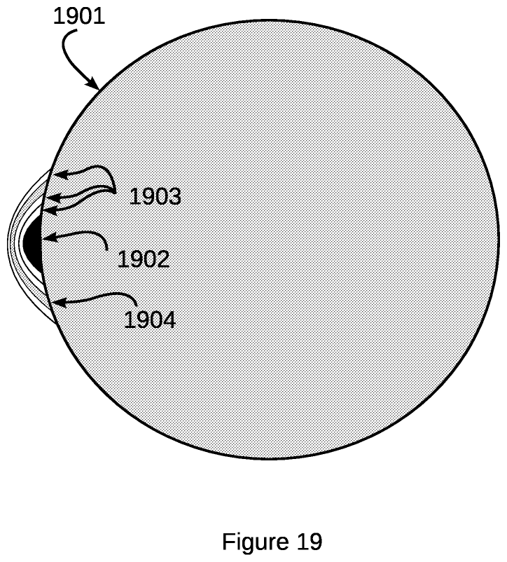

In one embodiment, a ball-lens test target is used to emulate an eye (FIG. 19).

In one embodiment of a ball-lens test target 1901, an absorptive spot (typically smaller than 500 microns in diameter) is deposited at the back surface of the ball-lens, emulating an optic nerve 1902. The spot is then coated with alternate layers of an optical adhesive or similar material, each layer having differing or alternating refractive index 1903, and together forming a backing structure with depth.

In one embodiment, one or more of the adhesive layers has embedded within it fluorescent particles 1904, intended for verifying fluorescence imaging.

In one embodiment, a ball-lens test target is mounted in an alignment tool 1121 which can be used in place of a mouse cartridge for alignment purposes.

In one embodiment, a ball-lens test target is mounted in a location typical for an eye in a toy mouse/phantom. The absorptive spot and layered backing structure are faced towards the interior of the toy mouse/phanom skull. This forms an assembly which can be mounted in a mouse holder cartridge and used as an alignment training aid.

In one embodiment, a tape-pin test target is used. This is formed by wrapping a length of pressure sensitive self adhesive tape around a pin former. This target has minimal variability in the spatial domain, yet has a well-defined layered structure formed from the varying refractive index provided by the alternating polymer tape and adhesive layers.

In one embodyment, a tape-pin test target is mounted for imaging on a rotatable objective crown. The rotation allows the pin to be aligned to a B-scan axis such that for the particular scan angle, there is minimal spatial variation in the axial position of the tape surface as resolved by OCT. This is used to characterize OCT depth resolving ability for the case where one axis has minimal spatial variation and an orthogonal B-scan axis has maximum variation.

In one embodiment, a tap-pin test target has fluorescent particles embedded between one or more layers. This can be used to characterize the fluorescence resolving performance.

### INDUSTRIAL APPLICATIONS

OCDR-OCT/SLO system and apparatus of this instant application is very useful for the diagnosis and management of ophthalmic diseases such as retinal diseases and glaucoma etc. Instant innovative OCDR-OCT/SLO diagnostic system leverages advancements in cross technological platforms. This enables us to supply the global market a very high resolution, robust OCDR-OCT/SLO imaging tool, which would be extremely useful to general physicians, optometrists and other health personnel.

The ophthalmic system and apparatus of this instant application is very useful for diagnosis and management of ophthalmic diseases such as retinal diseases and glaucoma etc. Instant innovative ophthalmic diagnostic system leverages advancements in cross technological platforms. This enables us to supply the global market a low-cost, portable, robust ophthalmic tool, which would be affordable to general physicians, optometrists and other health personnel.

It is to be understood that the embodiments described herein can be implemented in hardware, software or a combination thereof. For a hardware implementation, the embodiments (or modules thereof) can be implemented within one or more application specific integrated circuits (ASICs), mixed signal circuits, digital signal processors (DSPs), digital signal processing devices (DSPDs), programmable logic devices (PLDs), field programmable gate arrays (FPGAs), processors, graphical processing units (GPU), controllers, micro-controllers, microprocessors and/or other electronic units designed to perform the functions described herein, or a combination thereof.

For a mechanical hardware implementation, the embodiments (or modules thereof) can be implemented using various fabrication or prototyping methods.

When the embodiments (or partial embodiments) are implemented in software, firmware, middleware or microcode, program code or code segments, they can be stored in a machine-readable medium (or a computer-readable medium), such as a storage component. A code segment can represent a procedure, a function, a subprogram, a program, a routine, a subroutine, a module, a software package, a class, or any combination of instructions, data structures, or program statements. A code segment can be coupled to another code segment or a hardware circuit by passing and/or receiving information, data, arguments, parameters, or memory contents.

1. A system, comprising:
a light source emitting light of a specific bandwidth called a first light to acquire optical coherence tomography image;
the first light is sent to a specimen using a source arm and a sample arm;
a beam splitter to split the first light from the source arm as a first path light to a reference arm and as a second path light to the sample arm;
a mirror returning the first path light to the beam splitter to join a returning light from the specimen;
the sample arm sends the second path of light to the specimen through an objective and the specimen reflects back the second path of light as a returning light via the objective to the beam splitter;
a partial returning light from the beam splitter travels through a detector arm to a grating unit and a detector array;
the grating unit disperses the partial returning light from the beam splitter and a dispersed light enters the detector array to produce a light spectrum;
and a processor to perform a data analysis using an algorithm on the light spectrum to form an image of the specimen;
a second light source to illuminate the light on the specimen to acquire scanning laser ophthalmoscope image;
The second light source combining its path with the first light at a beam-splitter;
a lens to adjust the focus of the first light so that both the light sources focus at the same location at the specimen.

2. The system of claim 1, wherein there is a telescope to expand beams from both light sources and there is a pinhole within the telescope to eliminate stray light.

3. The system of claim 1, wherein a specimen is a part of an eye.

4. The system of claim 1, wherein the specific algorithm is at least one of frequency resampling, dispersion compensation, OCT angiography, adaptive optics and Doppler processing.

5. A system, comprising:
a tunable light source producing a light of various frequencies within a specific bandwidth called a first light;
the first light is sent to a specimen using a source arm and a sample arm;
a beam splitter to split the first light from the source arm as a first path light to a reference arm and as a second path light to the sample arm;
a mirror returning the first path light to the beam splitter to join a returning light from the specimen;
the sample arm sends the second path of light to the specimen through an objective and the specimen reflects back the second path of light as a returning light via the objective to the beam splitter;
a partial returning light from the beam splitter travels through the detector arm to a detector;
the detector to convert the partial returning light from the beam splitter into an electric current;
an analog to digital convertor to digitize the electric current into a digitized electric current; and
a processor to perform a data analysis using a specific algorithm on a digitized electric current to form an image of the specimen.

6. The system of claim 1, wherein the specific algorithm is at least one of frequency resampling, dispersion compensation, OCT angiography, adaptive optics and Doppler processing.

7. A positioner system comprising:
at least one translating stage to center the positioner system with respect to a core system,
at least one rotating device to rotate a specimen in its frame of reference;
at least one translating stage to move a specimen in a rotating frame of reference to align the specimen to the core system,
an alignment tool for centering positioner stages with respect to the core system,
so that rotating the specimen does not translate the specimen with respect to the core system that can perform at least one of measurements and therapy.

8. The system of claim 7 where the alignment tool centers the rotational axes of the positioner at a nodal point of the core system, and also aligns the specimen with the nodal point of the core system.

9. The system of claim 7 further utilizing a cartridge to rest the specimen on the positioner, and mounting features to deterministically position the cartridge on the positioner such that the specimen's eye is precisely positioned for the core system.

10. The system of claim 7 wherein the cartridge is angled so that the specimen axis is approximately aligned with the core system axis.

11. The system of claim 7 wherein the core system is an optical system wherein the optical system has an objective.

12. The system of claim 11 where the optical system performs at least one of signal detection and optical therapy.

13. The system of claim 12 wherein the optical system performs at least one of OCDR, OCT, OFDR, OCT angiography, Doppler OCT, spectroscopy, OCT spectroscopy, adaptive optics, fluorescence, reflectance, SLO measurements, therapy, electroretinogram measurements, optical surgery, laser surgery, electrical surgery, mechanical surgery, and chemical surgery.

14. The system of claim 7 wherein the specimen is at least one of a lens, fish, tadpole, mouse, rat, tree-shrew, rabbit, squirrel, squirrel monkey, a turtle, a reptile, a lizard and a snake.

15. The system of claim 9 wherein the cartridge is at least one of single-sided cartridges with a set of mounting features to precisely locate a specific eye, and ambidextrous with two sets of mounting features, each to precisely locate one specific eye.

16. The system of claim 9 wherein the cartridge has at least one of the features:
at least one orifice for gas anesthesia, a heater, a palate bar, a bite bar, and a muzzle strap;
furthermore at least one the features listed here translate and rotate with the cartridge.

17. The system of claim 7 further comprising at least one of alignment cameras, a sub-frame shared with the core system, a travel stop providing a repeatable position.

18. The system of claim 11 further comprising the optical system scanning mechanisms to create at least one of a line scan, orthogonal line scans, an XY scan, a raster scan, and a radial line scan.

19. The system of claim 18 further comprising cross-sectional OCT B-scans generated from the scanning mechanisms.

20. The system of claim 20 further comprising of OCT B-scans showing at least one of retinal and corneal contours and the positioner and the stages in the specimen's frame of reference are used to manipulate those retinal and corneal contours.
